(19) United States
(12) Patent Application Publication
(10) Pub. No.: US 2013/0260012 A1
(43) Pub. Date: Oct. 3, 2013

(54) INCREASED TUBER SET IN POTATO

(71) Applicant: J.R. SIMPLOT COMPANY, Boise, ID (US)

(72) Inventors: Caius M. Rommens, Boise, ID (US); Hua Yan, Boise, ID (US)

(73) Assignee: J.R. SIMPLOT COMPANY, Boise, ID (US)

(21) Appl. No.: 13/803,928

(22) Filed: Mar. 14, 2013

Related U.S. Application Data
(60) Provisional application No. 61/616,307, filed on Mar. 27, 2012.

(51) Int. Cl. C12N 15/82

(52) U.S. Cl. CPC C12N 15/8242
USPC 426/637; 800/290; 800/317.2; 435/320.1

(57) ABSTRACT

The present invention provides potato plant varieties with high tuber yield and tuber products with superior flavor and texture, and methods for increasing tuber yield and improving heat-processed product quality.

### CROSS-REFERENCE TO RELATED APPLICATIONS

This application claims the benefit of U.S. Provisional Patent Application No. 61/616,307, filed on Mar. 27, 2012, the contents of which are hereby incorporated by reference herein in their entirety.

### FIELD OF THE INVENTION

The field of the present inventive technology concerns methods and materials for increasing the number of tubers grown per potato plant by overexpressing and silencing certain genes involved in carotenoid formation.

### BACKGROUND OF THE INVENTION

The world's population has more than doubled in the last fifty years, multiplying from 3 billion to close to 7 billion, and it is projected to peak at more than 9 billion sometime in the next fifty or so years. See Word Population to 2300, Department of Economic and Social Affairs, United Nations (2004). The concomitant demand for food to feed the growing population is tremendous, so producing more food per acreage of farm land—such as by producing more edible vegetation per plant—would prove to be an incredibly advantageous way to help avert, or at least address, this mounting stress on resources.

The potato plant and potatoes are world dietary staples. Typically, a single cultivated commercial potato plant produces five to fifteen mature tubers. Increasing this yield-per-plant would be highly desirable.

A variety of factors, such genetics, physiology, and environmental conditions, induce potato plant stolons to “tuberize” into the thick starch-rich storage organs known as tubers. One important factor in the tuberization cycle is daylight: typically, wild potato plants will not tuberize to produce tubers if exposed to more than about 16 hours of daylight, but they will if the day is 12 or so hours long.

Because daylight is so important to tuberization, a number of related photoreceptive and photosensitive genes, as well as hormones, have been identified that are involved in this developmental process. One in particular, the photoreceptor PHYB, regulates tuber induction. When PHYB is silenced, however, the length of day, be it 12 or 16 or more hours, was found to have no effect on tuber set (Jackson et al., 1996). Accordingly, there have been many research efforts directed at increasing or decreasing the expression of proteins that interact directly with, or downstream of, PHYB.

Overexpression of genes such as the PHYB-inhibiting LK2 protein, for instance, or the PHYB responsive CO, mir172, StSP6A, and StBel5 proteins, results in altered or day length independent tuberization (Inui et al., 2010; Martinez-Garcia et al., 2002; Martin et al., 2009; Navarro et al., 2011; Chen et al., 2003). In most cases, this has been accomplished by inhibiting or activating the flowering locus T-like mobile signal tuberigen (Abelenda et al., 2010) and affecting levels of the plant hormone gibberellic acid (GA) (Jackson et al., 2000). Indeed, GA is known to play a dominant role in the timing of tuber formation (Xu et al., 1998). The alternative hormone abscisic acid (ABA) influences that timing by counteracting GA, whereas the regulating function of sucrose is caused by its effect on GA levels (Xu et al., 1998; Jackson, 1999). The positive influence of nitrogen withdrawal on the timing of tuber set was also linked to down-regulated amounts of GA and increases in ABA (Krauss, 1985). Hormones other than GA and ABA, including auxins, cytokinins, and jasmonic acid, do not seem to play a role in controlling the timing of tuber formation.

Such photoperiod sensitivity however was largely bred out of the cultivated potatoes used for commercial production in the United States. For cultivated potatoes, early flowering initiates tuberization, not daylight length, and subsequent bulking-up and maturation of tubers can take up to three months. Optimum moisture and nutrient levels early in the growing season, especially during the first 21 days after tuber emergence, are important to tuberization. Another important physiological variable for cultivated potatoes is the age of tubers that are used as seed: older seed produces more tubers than younger seed. Unlike in wild potato plants, little is known about the effects tuberigen and GA levels might have increasing tuber numbers in commercially-relevant cultivated potato plants.

There is an important need therefore to develop potato varieties that not only produce more tubers per plant but which also display all the sensory characteristics expected by consumers. The present invention creates and provides such new varieties, as well as the methods to develop them.

### SUMMARY OF THE INVENTION

In one embodiment, the invention provides a method for increasing tuber yield production in a potato plant comprising (A) overexpressing in a potato plant a neoxathin synthase gene and (B) downregulating in the same potato plant the expression of at least one of (i) cytochrome P450-type monooxygenase and (ii) zeaxanthin epoxidase, wherein the potato plant yields more mature tubers than a control potato plant. In an additional embodiment the inventionalso provides a method for increasing tuber yield production in a potato plant comprising down-regulating the expression of the chloroplast carotenoid epsilon-ring hydroxylase (ChxE) gene. Preferably, the potato plant is a variety selected from the group consisting of Bintje, Atlantic, Russet Burbank, Russet Ranger, Bondi and Moonlight. In a preferred aspect of the invention, (i) cytochrome P450-type monooxygenase and (ii) zeaxanthin epoxidase are both downregulated in the potato plant.

In a further embodiment, the invention provides a potato plant comprising in its genome an expression cassette for over-expressing a neoxathin synthase gene and at least one gene silencing expression cassette selected from the group consisting of (i) a gene silencing cassette for down-regulating cytochrome P450-type monooxygenase and (ii) a gene silencing cassette for down-regulating zeaxanthin epoxidase. Preferably, the potato plant genome comprises the two gene silencing cassettes of (i) and (ii). In a different embodiment the gene silencing cassette is for down-regulating the chloroplast carotenoid epsilon-ring hydroxylase, and the potato plant genome comprises all three gene silencing cassettes.

In a preferred aspect of the invention, the potato plant has an increased tuber yield production compared to a wild potato plant of the same variety. In an additional preferred aspect of the invention, the plant produces mature tubers having an average size of 26 to 38 mm.

In yet another embodiment, the invention provides a heat-processed product of the potato plant, wherein the heat-processed product has superior flavor, texture and appearance compared to a heat-processed product of a wild potato plant of the same variety. Preferably, the heat-processed product is a French fry or a roasted potato containing up to 30% of the oil content of a French fry or roasted potato of a wild potato plant of the same variety.

In a further embodiment, the invention provides a vector comprising (A) an expression cassette for expressing a neoxathin synthase gene; and (B) at least one gene silencing expression cassette selected from the group consisting of (i) a gene silencing cassette for down-regulating cytochrome P450-type monooxygenase and (ii) a gene silencing cassette for down-regulating zeaxanthin epoxidase. In a preferred aspect, the vector comprises both gene silencing cassettes of (i) and (ii).

In another embodiment, the invention provides a method for increasing tuber yield production in a potato plant comprising over-expressing in a potato plant a phytoetene synthase gene and down-regulating in the same potato plant the expression of at least one of (i) de-etiolated homolog 1, (ii) carotenoid dioxygenase 1B and (iii) cytochrome P450-type monooxygenase, wherein the potato plant yields more mature tubers than a control potato plant. In a preferred aspect of the invention, the potato plant is a variety selected from the group consisting of Bintje, Atlantic, Russet Burbank, Russet Ranger, Bondi and Moonlight. Preferably, (i) de-etiolated homolog 1, (ii) carotenoid dioxygenase 1B and (iii) cytochrome P450-type monooxygenase are all down-regulated in the potato plant.

In an additional embodiment, the invention provides a potato plant comprising in its genome an expression cassette for over-expressing a phytoetene synthase gene and at least one gene silencing expression cassette selected from the group consisting of (i) a gene silencing cassette for down-regulating de-etiolated homolog 1, (ii) a gene silencing cassette for down-regulating carotenoid dioxygenase 1B and (iii) a gene silencing cassette for down-regulating cytochrome P450-type monooxygenase. In a preferred aspect of the invention, the genome of the potato plant comprises all three gene silencing cassettes of (i), (ii) and (iii). In another preferred aspect of the invention, the potato plant has an increased tuber yield production compared to a wild potato plant of the same variety. Preferably, the potato plant produces mature tubers having an average size of 26 to 38 mm.

In a further embodiment, the invention provides a heat-processed product of the potato plant, wherein the heat-processed product has superior flavor, texture and appearance compared to a heat-processed product of a wild potato plant of the same variety. Preferably, the heat-processed product is a French fry or a roasted potato containing up to 30% of the oil content of a French fry or roasted potato of a wild potato plant of the same variety.

In yet another embodiment, the invention provides a vector comprising (A) an expression cassette for expressing a phytoetene synthase gene; and (B) at least one gene silencing expression cassette selected from the group consisting of (i) a gene silencing cassette for down-regulating de-etiolated homolog 1, (ii) a gene silencing cassette for down-regulating carotenoid dioxygenase 1B and (iii) a gene silencing cassette for down-regulating cytochrome P450-type monooxygenase. In a preferred aspect of the invention, the vector comprises all three gene silencing cassettes of (i), (ii) and (iii).

### BRIEF DESCRIPTION OF THE DRAWINGS

This application contains at least one drawing executed in color.

FIG. 1 illustrates the plasmid map of pSIM2063 for silencing the GA20ox1 gene.

FIG. 2 illustrates the plasmid map of pSIM2064 for silencing StCYP and StZep, and for overexpressing StNXS2m.

FIG. 3 shows Bintje versus IO7-11G tubers.

FIG. 4 shows a Southern blot of the IO7-11G transformed Bintje line with over expressed genes.

FIG. 5 shows a Northern blot of the IO7-11G transformed Bintje line with ZmPsy and StDXS 1 probes.

FIG. 6 shows a Southern blot of the IO7-11G transformed Bintje line with silenced genes.

FIG. 7 shows a semi-quantitative Reverse Transcriptase-PCR of the IO7-11G transformed Bintje line with silenced genes.

### DETAILED DESCRIPTION OF THE INVENTION

A number of studies describe the ability to increase tuber set on potatoes upon the manipulation of one or few genes. These studies center on genes involved in hormone synthesis or perception, light quality or duration perception and starch synthesis and partitioning.

A strong correlation exists in potatoes between decreased levels of GA activity and tuber initiation. Gibberellic Acids (GAs) have an inhibitory effect on tuberization. Gibberellin activity decreases under conditions that promote tuberization such as short days (SD) (Kumar & Wareing 1974; Railton & Wareing 1973) and increases in plants subjected to conditions which inhibit tuberization (Krauss & Marschner 1982; Menzel 1983). Decreased levels of GA1 are observed in stolon tips during the early stages of tuberization (Xu et al. 1998).

GAs are biosynthesized from geranylgeranyl diphosphate, a common C20 precursor for diterpenoids. Conversions of geranylgeranyl diphosphate into bioactive GAs, such as GA1 and GA4, involve three classes of enzymes: plastid-localized terpene cyclases, membrane-bound cytochrome P450 monooxygenases (P450s), and soluble 2-oxoglutarate-dependent dioxygenases (2ODDs). The expression of GA 20-oxidase and GA 3β-hydroxylase, two enzymes that catalyze the two last steps in GA biosynthetic pathway, is subject to feedback regulation by the pathway end-product GA1 (Chiang et al. 1995; Phillips et al. 1995). GA 20-oxidase expression is regulated by light, with significantly higher levels of transcript detected in long-day (LD) as compared to SD conditions in both spinach and Arabidopsis plants (Wu et al. 1996; Xu et al. 1995).

It has been reported that transgenic potato lines with reduced levels of expression of the StGA20ox1 mRNA have shorter stems relative to controls, and, when grown under SD conditions, tuberize earlier and have a higher tuber yield than the controls. However, the tubers formed directly on the stem and not on the stolons (Carrera et al. 2000).

A different pattern of tuberization is exhibited by the andigena transformants bearing an antisense construct for the phytochrome phyB gene (Jackson et al. 1996). These plants tuberize equally well under inductive and non-inductive conditions, (Jackson & Prat 1996; Jackson et al. 1996), and readily form tubers after 1 month under LD conditions.

The Snf1/AMP-activated protein kinase (AMPK) family is essential for metabolic regulation in eukaryotes. The SNF1-homologue in plants, SnRK1, regulates carbon metabolism through both gene expression and direct control of enzyme activity. Antisense expression of a SnRK1 sequence in potato resulted in the loss of sucrose-inducibility of sucrose synthase gene expression in leaves and in the reduction of sucrose synthase gene expression in tubers (Purcell, Smith & Halford 1998).

Transgenic potato plants that were constitutively silenced for a gene encoding the SnRK-interacting protein GAL83 (StGal83) were reported to produce more tubers when grown in vitro or in growth chambers, possibly by altering the metabolic status of leaves (Lovas et al., 2003). It also appeared possible to increase the number of tubers produced per plant in the greenhouse by constitutively silencing the cytosolic phosphorylase (PhH) gene (Duwenig et al., 1997); transgenic plants seemed to yield 1.6 to 2.4 fold more tubers than untransformed controls. The greenhouse-based efficacy of the StGal83 and PhH gene silencing approaches could not however be reproduced in the field (see Examples 1 and 2).

Carotenoids are plant pigments that function as antioxidants, hormone precursors, colorants and essential components of the photosynthetic apparatus, and, since they accumulate in nearly all types of plastids, not just the chloroplast, they are found in most plant organs and tissues. Potato tubers accumulate primarily β-cryptoxanthin or lutein and appear white or pale yellow, although potatoes with orange flesh were found in cultivated white-flesh potato populations and the orange was associated with large amounts of zeaxanthin.

Xanthophylls typically have either a hydroxy at C-3 or an epoxy at the 5,6-position of the ionone ring. Hydroxylation of the β- and ε-rings are carried out by different enzymes: β-hydroxylase (β-OH) acts on β rings and ε-hydroxylase (ε-OH) acts on ε rings. The ε-OH is a cytochrome P450-type monooxygenase and differs from β-hydroxylase, which is a non-haeme diiron monooxygenase. The action of these two enzymes in the β,ε branch results in the formation of lutein, a 3,3′-dihydroxy-α-carotene. In the β,β branch β-OH acts in two steps to produce β-cryptoxanthin and then zeaxanthin, a 3,3′-dihydroxy-β-carotene. Lutein is the end product of the β,ε branch, whereas zeaxanthin can be further modified by epoxidation to produce violaxanthin. Under high light stress, violaxanthin de-epoxidase (VDE) catalyses the de-epoxidation of violaxanthin back to zeaxanthin. Violaxanthin is converted to neoxanthin by neoxanthin synthase (NXS). Neoxanthin is the last carotenoid of the β,β branch of the carotenoid pathway in higher plants.

Cytochrome P450 enzymes (CYPs) constitute a large superfamily of heme-containing monooxygenases that are widely distributed in all kingdoms of life and are involved in the metabolism of a wide variety of endogenous and xenobiotic compounds by catalyzing regio- and stereospecific monooxygenation with an oxygen atom generated from molecular oxygen. A common feature to these enzymes is their sensitivity to environmental factors, including light.

Potato plant varieties present wide differences in texture and flavors. Highly desirable potato varieties include, among others, the Bintje, Atlantic, Russet Burbank, Ranger Russet, Bondi and Moonlight varieties.

Bintje potatoes are the most widely grown yellow-fleshed potato, present tolerance to a wide range of soils and are commercially appreciated for their storage properties, good looks, silky skin and remarkable flavor.

Atlantic potatoes are known for their attractive tubers and high quality chips.

The Russett Burbank potato variety is the major cultivar grown in the United States and is widely used for French fries and baking.

Ranger Russet is full-season potato variety, which produces a large yield of high quality, long, russet-skinned tubers that are well suited for baking and processing into French fries.

The Bondi variety is suitable as a storage French fry potato.

“Moonlight” is a crop potato cultivar with high yield potential that has been developed for the fresh market as well as for French fry production.

It would be highly desirable to increase tuber yield of these potato varieties, while improving their texture and flavors. The present invention satisfies this need by providing potato plant varieties with high tuber yield and tuber products with superior flavor and texture, and the methods for increasing tuber yield and improving heat-processed product quality. Thus, the present invention provides whole miniature potato bakers obtained from the most desirable potato plant varieties. The “baby bakers” of the invention have delicate skins, buttery yellow flesh and exceptional flavor, texture and appearance. The presence of the skin enhances hold during baking or frying, and prevents excessive oil absorption upon cooking Accordingly, baby baker French fries retain 20-30% of oil when compared to French fries of regular size potatoes.

The present invention uses terms and phrases that are well known to those practicing the art. Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described herein are those well known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. The techniques and procedures are generally performed according to conventional methodology (Molecular Cloning, A Laboratory Manual, 3rd. edition, edited by Sambrook & Russel Cold Spring Harbor Laboratory Press, Cold Spring Harbor, N.Y., 2001).

Agrobacterium or bacterial transformation: as is well known in the field, Agrobacteria that are used for transforming plant cells are disarmed and virulent derivatives of, usually, Agrobacterium tumefaciens or Agrobacterium rhizogenes. Upon infection of plants, explants, cells, or protoplasts, the Agrobacterium transfers a DNA segment from a plasmid vector to the plant cell nucleus. The vector typically contains a desired polynucleotide that is located between the borders of a T-DNA. However, any bacteria capable of transforming a plant cell may be used, such as, Rhizobium trifolii, Rhizobium leguminosarum, Phyllobacterium myrsinacearum, SinoRhizobium meliloti, and MesoRhizobium loti.

Angiosperm: vascular plants having seeds enclosed in an ovary. Angiosperms are seed plants that produce flowers that bear fruits. Angiosperms are divided into dicotyledonous and monocotyledonous plant.

Antibiotic Resistance: ability of a cell to survive in the presence of an antibiotic. Antibiotic resistance, as used herein, results from the expression of an antibiotic resistance gene in a host cell. A cell may have resistance to any antibiotic. Examples of commonly used antibiotics include kanamycin and hygromycin.

Dicotyledonous plant (dicot): a flowering plant whose embryos have two seed halves or cotyledons, branching leaf veins, and flower parts in multiples of four or five. Examples of dicots include but are not limited to, potato, sugar beet, broccoli, cassava, sweet potato, pepper, poinsettia, bean, alfalfa, soybean, and avocado.

Endogenous: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. Expression cassette: polynucleotide comprising, from 5′ to 3′, (a) a first promoter, (b) a sequence comprising (i) at least one copy of a gene or gene fragment, or (ii) at least one copy of a fragment of the promoter of a gene, and (c) either a terminator or a second promoter that is positioned in the opposite orientation as the first promoter.

Foreign: “foreign,” with respect to a nucleic acid, means that that nucleic acid is derived from non-plant organisms, or derived from a plant that is not the same species as the plant to be transformed or is not derived from a plant that is not interfertile with the plant to be transformed, does not belong to the species of the target plant. According to the present invention, foreign DNA or RNA represents nucleic acids that are naturally occurring in the genetic makeup of fungi, bacteria, viruses, mammals, fish or birds, but are not naturally occurring in the plant that is to be transformed. Thus, a foreign nucleic acid is one that encodes, for instance, a polypeptide that is not naturally produced by the transformed plant. A foreign nucleic acid does not have to encode a protein product.

Gene: A gene is a segment of a DNA molecule that contains all the information required for synthesis of a product, polypeptide chain or RNA molecule that includes both coding and non-coding sequences. A gene can also represent multiple sequences, each of which may be expressed independently, and may encode slightly different proteins that display the same functional activity. For instance, the asparagine synthetase 1 and 2 genes can, together, be referred to as a gene.

Genetic element: a “genetic element” is any discreet nucleotide sequence such as, but not limited to, a promoter, gene, terminator, intron, enhancer, spacer, 5′-untranslated region, 3′-untranslated region, or recombinase recognition site.

Genetic modification: stable introduction of DNA into the genome of certain organisms by applying methods in molecular and cell biology.

Gymnosperm: as used herein, refers to a seed plant that bears seed without ovaries. Examples of gymnosperms include conifers, cycads, ginkgos, and ephedras.

Introduction: as used herein, refers to the insertion of a nucleic acid sequence into a cell, by methods including infection, transfection, transformation or transduction.

Monocotyledonous plant (monocot): a flowering plant having embryos with one cotyledon or seed leaf, parallel leaf veins, and flower parts in multiples of three. Examples of monocots include, but are not limited to maize, rice, oat, wheat, barley, and sorghum.

Native: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Native DNA: any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present invention. For instance, a native DNA may comprise a point mutation since such point mutations occur naturally. It is also possible to link two different native DNAs by employing restriction sites because such sites are ubiquitous in plant genomes.

Native Nucleic Acid Construct: a polynucleotide comprising at least one native DNA.

Operably linked: combining two or more molecules in such a fashion that in combination they function properly in a plant cell. For instance, a promoter is operably linked to a structural gene when the promoter controls transcription of the structural gene.

Overexpression: expression of a gene to levels that are higher than those in control plants.

P-DNA: a plant-derived transfer-DNA (“P-DNA”) border sequence is not identical in nucleotide sequence to any known bacterium-derived T-DNA border sequence, but it functions for essentially the same purpose. That is, the P-DNA can be used to transfer and integrate one polynucleotide into another. A P-DNA can be inserted into a tumor-inducing plasmid, such as a Ti-plasmid from Agrobacterium in place of a conventional T-DNA, and maintained in a bacterium strain, just like conventional transformation plasmids. The P-DNA can be manipulated so as to contain a desired polynucleotide, which is destined for integration into a plant genome via bacteria-mediated plant transformation. The P-DNA comprises at least one border sequence. See Rommens et al. 2005 Plant Physiology 139: 1338-1349, which is incorporated herein by reference. In certain embodiments of the invention, the T-DNA is replaced by the P-DNA.

Phenotype: phenotype is a distinguishing feature or characteristic of a plant, which may be altered according to the present invention by integrating one or more “desired polynucleotides” and/or screenable/selectable markers into the genome of at least one plant cell of a transformed plant. The “desired polynucleotide(s)” and/or markers may confer a change in the phenotype of a transformed plant, by modifying any one of a number of genetic, molecular, biochemical, physiological, morphological, or agronomic characteristics or properties of the transformed plant cell or plant as a whole.

Plant tissue: a “plant” is any of various photosynthetic, eukaryotic, multicellular organisms of the kingdom Plantae characteristically producing embryos, containing chloroplasts, and having cellulose cell walls. A part of a plant, i.e., a “plant tissue” may be treated according to the methods of the present invention to produce a transgenic plant. Many suitable plant tissues can be transformed according to the present invention and include, but are not limited to, somatic embryos, pollen, leaves, stems, calli, stolons, microtubers, and shoots. Thus, the present invention envisions the transformation of angiosperm and gymnosperm plants such as wheat, maize, rice, barley, oat, sugar beet, potato, tomato, alfalfa, cassaya, sweet potato, and soybean. According to the present invention “plant tissue” also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed. Of particular interest are potato, maize, and wheat.

Plant transformation and cell culture: broadly refers to the process by which plant cells are genetically modified and transferred to an appropriate plant culture medium for maintenance, further growth, and/or further development. Such methods are well known to the skilled artisan.

Processing: the process of producing a food from (1) the seed of, for instance, wheat, corn, coffee plant, or cocoa tree, (2) the tuber of, for instance, potato, or (3) the root of, for instance, sweet potato and yam comprising heating to at least 120° C. Examples of processed foods include bread, breakfast cereal, pies, cakes, toast, biscuits, cookies, pizza, pretzels, tortilla, French fries, oven-baked fries, potato chips, hash browns, roasted coffee, and cocoa.

Progeny: a “progeny” of the present invention, such as the progeny of a transgenic plant, is one that is born of, begotten by, or derived from a plant or the transgenic plant. Thus, a “progeny” plant, i.e., an “F1” generation plant is an offspring or a descendant of the transgenic plant produced by the inventive methods. A progeny of a transgenic plant may contain in at least one, some, or all of its cell genomes, the desired polynucleotide that was integrated into a cell of the parent transgenic plant by the methods described herein. Thus, the desired polynucleotide is “transmitted” or “inherited” by the progeny plant. The desired polynucleotide that is so inherited in the progeny plant may reside within a T-DNA construct, which also is inherited by the progeny plant from its parent. The term “progeny” as used herein, also may be considered to be the offspring or descendants of a group of plants.

Promoter: promoter is intended to mean a nucleic acid, preferably DNA that binds RNA polymerase and/or other transcription regulatory elements. As with any promoter, the promoters of the current invention will facilitate or control the transcription of DNA or RNA to generate an mRNA molecule from a nucleic acid molecule that is operably linked to the promoter. As stated earlier, the RNA generated may code for a protein or polypeptide or may code for an RNA interfering, or antisense molecule.

A promoter is a nucleic acid sequence that enables a gene with which it is associated to be transcribed. In prokaryotes, a promoter typically consists of two short sequences at −10 and −35 position upstream of the gene, that is, prior to the gene in the direction of transcription. The sequence at the −10 position is called the Pribnow box and usually consists of the six nucleotides TATAAT. The Pribnow box is essential to start transcription in prokaryotes. The other sequence at −35 usually consists of the six nucleotides TTGACA, the presence of which facilitates the rate of transcription.

Eukaryotic promoters are more diverse and therefore more difficult to characterize, yet there are certain fundamental characteristics. For instance, eukaryotic promoters typically lie upstream of the gene to which they are most immediately associated. Promoters can have regulatory elements located several kilobases away from their transcriptional start site, although certain tertiary structural formations by the transcriptional complex can cause DNA to fold, which brings those regulatory elements closer to the actual site of transcription. Many eukaryotic promoters contain a “TATA box” sequence, typically denoted by the nucleotide sequence, TATAAA. This element binds a TATA binding protein, which aids formation of the RNA polymerase transcriptional complex. The TATA box typically lies within 50 bases of the transcriptional start site.

Eukaryotic promoters also are characterized by the presence of certain regulatory sequences that bind transcription factors involved in the formation of the transcriptional complex. An example is the E-box denoted by the sequence CACGTG, which binds transcription factors in the basic-helix-loop-helix family. There also are regions that are high in GC nucleotide content.

Hence, according to the present invention, a partial sequence, or a specific promoter “fragment” of a promoter that may be used in the design of a desired polynucleotide of the present invention may or may not comprise one or more of these elements or none of these elements. In one embodiment, a promoter fragment sequence of the present invention is not functional and does not contain a TATA box.

The desired polynucleotide may be linked in two different orientations to the promoter. In one orientation, e.g., “sense”, at least the 5′-part of the resultant RNA transcript will share sequence identity with at least part of at least one target transcript. In the other orientation designated as “antisense”, at least the 5′-part of the predicted transcript will be identical or homologous to at least part of the inverse complement of at least one target transcript.

A plant promoter is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as xylem, leaves, roots, or seeds. Such promoters are referred to as tissue-preferred promoters. Promoters which initiate transcription only in certain tissues are referred to as tissue-specific promoters. A cell type-specific promoter primarily drives expression in certain cell types in one or more organs, for example, vascular cells in roots or leaves. An inducible or repressible promoter is a promoter which is under environmental control. Examples of environmental conditions that may effect transcription by inducible promoters include anaerobic conditions or the presence of light. Tissue specific, tissue preferred, cell type specific, and inducible promoters constitute the class of non-constitutive promoters. A constitutive promoter is a promoter which is active under most environmental conditions, and in most plant parts.

Polynucleotide is a nucleotide sequence, comprising a gene coding sequence or a fragment thereof, (comprising at least 15 consecutive nucleotides, preferably at least 30 consecutive nucleotides, and more preferably at least 50 consecutive nucleotides), a promoter, an intron, an enhancer region, a polyadenylation site, a translation initiation site, 5′ or 3′ untranslated regions, a reporter gene, a selectable marker or the like. The polynucleotide may comprise single stranded or double stranded DNA or RNA. The polynucleotide may comprise modified bases or a modified backbone. The polynucleotide may be genomic, an RNA transcript (such as an mRNA) or a processed nucleotide sequence (such as a cDNA). The polynucleotide may comprise a sequence in either sense or antisense orientations.

An isolated polynucleotide is a polynucleotide sequence that is not in its native state, e.g., the polynucleotide is comprised of a nucleotide sequence not found in nature or the polynucleotide is separated from nucleotide sequences with which it typically is in proximity or is next to nucleotide sequences with which it typically is not in proximity.

Seed: a “seed” may be regarded as a ripened plant ovule containing an embryo, and a propagative part of a plant, as a tuber or spore. Seed may be incubated prior to Agrobacterium-mediated transformation, in the dark, for instance, to facilitate germination. Seed also may be sterilized prior to incubation, such as by brief treatment with bleach. The resultant seedling can then be exposed to a desired strain of Agrobacterium.

Selectable/screenable marker: a gene that, if expressed in plants or plant tissues, makes it possible to distinguish them from other plants or plant tissues that do not express that gene. Screening procedures may require assays for expression of proteins encoded by the screenable marker gene. Examples of selectable markers include the neomycin phosphotransferase (NptII) gene encoding kanamycin and geneticin resistance, the hygromycin phosphotransferase (HptII) gene encoding resistance to hygromycin, or other similar genes known in the art.

Sensory characteristics: panels of professionally trained individuals can rate food products for sensory characteristics such as appearance, flavor, aroma, and texture. Thus, the present invention contemplates improving the sensory characteristics of a plant product obtained from a plant that has been modified according to the present invention to manipulate its tuber yield production.

Sequence identity: as used herein, “sequence identity” or “identity” in the context of two nucleic acid or polypeptide sequences includes reference to the residues in the two sequences which are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11 17 (1988) e.g., as implemented in the program PC/GENE (Intelligenetics, Mountain View, Calif., USA).

As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Sequence identity” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS IN MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in GUIDE TO HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215: 403 (1990)), and FASTDB (Brutlag et al., Comp. App. Biosci. 6: 237 (1990)).

Silencing: The unidirectional and unperturbed transcription of either genes or gene fragments from promoter to terminator can trigger post-transcriptional silencing of target genes. Initial expression cassettes for post-transcriptional gene silencing in plants comprised a single gene fragment positioned in either the antisense (McCormick et al., U.S. Pat. No. 6,617,496; Shewmaker et al., U.S. Pat. No. 5,107,065) or sense (van der Krol et al., Plant Cell 2:291-299, 1990) orientation between regulatory sequences for transcript initiation and termination. In Arabidopsis, recognition of the resulting transcripts by RNA-dependent RNA polymerase leads to the production of double-stranded (ds) RNA. Cleavage of this dsRNA by Dicer-like (Dcl) proteins such as Dc14 yields 21-nucleotide (nt) small interfering RNAs (siRNAs). These siRNAs complex with proteins including members of the Argonaute (Ago) family to produce RNA-induced silencing complexes (RISCs). The RISCs then target homologous RNAs for endonucleolytic cleavage.

More effective silencing constructs contain both a sense and antisense component, producing RNA molecules that fold back into hairpin structures (Waterhouse et al., Proc Natl Acad Sci USA 95: 13959-13964, 1998). The high dsRNA levels produced by expression of inverted repeat transgenes were hypothesized to promote the activity of multiple Dcls. Analyses of combinatorial Dcl knockouts in Arabidopsis supported this idea, and also identified Dcl4 as one of the proteins involved in RNA cleavage.

One component of conventional sense, antisense, and double-strand (ds) RNA-based gene silencing constructs is the transcriptional terminator. WO 2006/036739, which is incorporated in its entirety by reference, shows that this regulatory element becomes obsolete when gene fragments are positioned between two oppositely oriented and functionally active promoters. The resulting convergent transcription triggers gene silencing that is at least as effective as unidirectional ‘promoter-to-terminator’ transcription. In addition to short variably-sized and non-polyadenylated RNAs, terminator-free cassette produced rare longer transcripts that reach into the flanking promoter. Replacement of gene fragments by promoter-derived sequences further increased the extent of gene silencing.

In a preferred embodiment of the present invention, the desired polynucleotide comprises a partial sequence of a target gene promoter or a partial sequence that shares sequence identity with a portion of a target gene promoter. Hence, a desired polynucleotide of the present invention contains a specific fragment of a particular target gene promoter of interest.

The desired polynucleotide may be operably linked to one or more functional promoters. Various constructs contemplated by the present invention include, but are not limited to (1) a construct where the desired polynucleotide comprises one or more promoter fragment sequences and is operably linked at both ends to functional ‘driver’ promoters. Those two functional promoters are arranged in a convergent orientation so that each strand of the desired polynucleotide is transcribed; (2) a construct where the desired polynucleotide is operably linked to one functional promoter at either its 5′-end or its 3′-end, and the desired polynucleotide is also operably linked at its non-promoter end by a functional terminator sequence; (3) a construct where the desired polynucleotide is operably linked to one functional promoter at either its 5′-end or its 3′-end, but where the desired polynucleotide is not operably linked to a terminator; or (4) a cassette, where the desired polynucleotide comprises one or more promoter fragment sequences but is not operably linked to any functional promoters or terminators.

Hence, a construct of the present invention may comprise two or more ‘driver’ promoters which flank one or more desired polynucleotides or which flank copies of a desired polynucleotide, such that both strands of the desired polynucleotide are transcribed. That is, one promoter may be oriented to initiate transcription of the 5′-end of a desired polynucleotide, while a second promoter may be operably oriented to initiate transcription from the 3′-end of the same desired polynucleotide. The oppositely-oriented promoters may flank multiple copies of the desired polynucleotide. Hence, the “copy number” may vary so that a construct may comprise 2, 3, 4, 5, 6, 7, 8, 9, 10, 15, 20, 30, 40, 50, 60, 70, 80, 90, or 100, or more than 100 copies, or any integer in-between, of a desired polynucleotide, which may be flanked by the ‘driver’ promoters that are oriented to induce convergent transcription. If neither cassette comprises a terminator sequence, then such a construct, by virtue of the convergent transcription arrangement, may produce RNA transcripts that are of different lengths. In this situation, therefore, there may exist subpopulations of partially or fully transcribed RNA transcripts that comprise partial or full-length sequences of the transcribed desired polynucleotide from the respective cassette. Alternatively, in the absence of a functional terminator, the transcription machinery may proceed past the end of a desired polynucleotide to produce a transcript that is longer than the length of the desired polynucleotide.

In a construct that comprises two copies of a desired polynucleotide, therefore, where one of the polynucleotides may or may not be oriented in the inverse complementary direction to the other, and where the polynucleotides are operably linked to promoters to induce convergent transcription, and there is no functional terminator in the construct, the transcription machinery that initiates from one desired polynucleotide may proceed to transcribe the other copy of the desired polynucleotide and vice versa. The multiple copies of the desired polynucleotide may be oriented in various permutations: in the case where two copies of the desired polynucleotide are present in the construct, the copies may, for example, both be oriented in same direction, in the reverse orientation to each other, or in the inverse complement orientation to each other, for example.

In an arrangement where one of the desired polynucleotides is oriented in the inverse complementary orientation to the other polynucleotide, an RNA transcript may be produced that comprises not only the “sense” sequence of the first polynucleotide but also the “antisense” sequence from the second polynucleotide. If the first and second polynucleotides comprise the same or substantially the same DNA sequences, then the single RNA transcript may comprise two regions that are complementary to one another and which may, therefore, anneal. Hence, the single RNA transcript that is so transcribed, may form a partial or full hairpin duplex structure.

On the other hand, if two copies of such a long transcript were produced, one from each promoter, then there will exist two RNA molecules, each of which would share regions of sequence complementarity with the other. Hence, the “sense” region of the first RNA transcript may anneal to the “antisense” region of the second RNA transcript and vice versa. In this arrangement, therefore, another RNA duplex may be formed which will consist of two separate RNA transcripts, as opposed to a hairpin duplex that forms from a single self-complementary RNA transcript.

Alternatively, two copies of the desired polynucleotide may be oriented in the same direction so that, in the case of transcription read-through, the long RNA transcript that is produced from one promoter may comprise, for instance, the sense sequence of the first copy of the desired polynucleotide and also the sense sequence of the second copy of the desired polynucleotide. The RNA transcript that is produced from the other convergently-oriented promoter, therefore, may comprise the antisense sequence of the second copy of the desired polynucleotide and also the antisense sequence of the first polynucleotide. Accordingly, it is likely that neither RNA transcript would contain regions of exact complementarity and, therefore, neither RNA transcript is likely to fold on itself to produce a hairpin structure. On the other hand the two individual RNA transcripts could hybridize and anneal to one another to form an RNA duplex.

Tissue: any part of a plant that is used to produce a food. A tissue can be a tuber of a potato, a root of a sweet potato, or a seed of a maize plant.

Transcriptional terminators: The expression DNA constructs of the present invention typically have a transcriptional termination region at the opposite end from the transcription initiation regulatory region. The transcriptional termination region may be selected, for stability of the mRNA to enhance expression and/or for the addition of polyadenylation tails added to the gene transcription product. Translation of a nascent polypeptide undergoes termination when any of the three chain-termination codons enters the A site on the ribosome. Translation termination codons are UAA, UAG, and UGA. In the instant invention, transcription terminators are derived from either a gene or, more preferably, from a sequence that does not represent a gene but intergenic DNA. For example, the terminator sequence from the potato ubiquitin gene may be used.

Transfer DNA (T-DNA): a transfer DNA is a DNA segment delineated by T-DNA borders borders to create a T-DNA. A T-DNA is a genetic element that is well-known as an element capable of integrating a nucleotide sequence contained within its borders into another genome. In this respect, a T-DNA is flanked, typically, by two “border” sequences. A desired polynucleotide of the present invention and a selectable marker may be positioned between the left border-like sequence and the right border-like sequence of a T-DNA. The desired polynucleotide and selectable marker contained within the T-DNA may be operably linked to a variety of different, plant-specific (i.e., native), or foreign nucleic acids, like promoter and terminator regulatory elements that facilitate its expression, i.e., transcription and/or translation of the DNA sequence encoded by the desired polynucleotide or selectable marker.

Transformation of plant cells: A process by which a nucleic acid is stably inserted into the genome of a plant cell. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols such as ‘refined transformation’ or ‘precise breeding’, viral infection, whiskers, electroporation, microinjection, polyethylene glycol-treatment, heat shock, lipofection and particle bombardment.

Transgenic plant: a transgenic plant of the present invention is one that comprises at least one cell genome in which an exogenous nucleic acid has been stably integrated. According to the present invention, a transgenic plant is a plant that comprises only one genetically modified cell and cell genome, or is a plant that comprises some genetically modified cells, or is a plant in which all of the cells are genetically modified. A transgenic plant of the present invention may be one that comprises expression of the desired polynucleotide, i.e., the exogenous nucleic acid, in only certain parts of the plant. Thus, a transgenic plant may contain only genetically modified cells in certain parts of its structure.

Variant: a “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. “Variant” may also refer to a “shuffled gene” such as those described in Maxygen-assigned patents.

It is understood that the present invention is not limited to the particular methodology, protocols, vectors, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a gene” is a reference to one or more genes and includes equivalents thereof known to those skilled in the art and so forth. Indeed, one skilled in the art can use the methods described herein to express any native gene (known presently or subsequently) in plant host systems.

The following examples are set forth as representative of specific and preferred embodiments of the present invention. These examples are not to be construed as limiting the scope of the invention in any manner. It should be understood that many variations and modifications can be made while remaining within the spirit and scope of the invention.

### EXAMPLES

The following studies were undertaken to better understand the interaction of phyB signaling and GA metabolism and their function in regulation of potato tuber development.

### Example 1

### StGal83 Gene Silencing does not Increase Tuber Quantity in the Field

The Gal83 gene encodes the beta-subunit of a protein kinase complex that is modulated by changes in the cellular AMT/ATP ratio. It is an important regulator of the plant's metabolic and stress response. In the potato variety “White Lady”, antisense repression of Gal83 had been reported to increase the number of tubers produced per plant from an average of 2.1 for controls to 2.9-3.5 for transgenic lines (Lovas et al., Plant J 33: 139-147, 2003). In an attempt to confirm this finding for the variety “Bintje”, plants were transformed with a transfer DNA carrying a silencing cassette with two fragments of the potato Gal83 (StGal83) gene (see SEQ ID NO:1 for the StGal83 cDNA and SEQ ID NO:2 and 3 for the fragments used in silencing), positioned as inverted repeats between the strong promoter of the ADP glucose pyrophosphorylase gene (AGP) (SEQ ID NO:4 gives the promoter sequence) and the terminator of the Ubiquitin-3 gene (Ubi3) (SEQ ID NO:5 for terminator) (pSIM1448). Control plants were obtained through transformation with a transfer DNA containing only a selectable marker gene (pSIM401, see Rommens et al., 2005). Transgenic plants were propagated to produce lines, and planted in the greenhouse in 1-gallon pots (Table 1).

The experiment was repeated with three copies of each of the four best lines (1448-13, 15, 21, 24) in 2-gallon pots. RNA extracted from leaf tissues was then hybridized with a 1048-bp probe derived from a cDNA of the targeted gene (SEQ ID NO:6). The StGAL83 transcript was clearly present in control lines but absent from lines 1448-13, 15, 21, and 24. However, StGal83 gene silencing was not correlated with an increase in the number of tubers produced per plant. Lines 15, 21, and 24 yielded the same number of tubers as controls which was, on average, 12-18 tubers/plant. Line 13 appeared to produce about twice as many tubers/plant (Table 2) but this line did not contain lower StGal83 transcript levels than the other three lines, indicating that the increased number of tubers should be considered an effect of somaclonal variation. To determine the number of tubers that could be obtained outside, five greenhouse-grown tubers from each of the four transgenic lines plus control plants were planted in the field. Tubers from additional 1448 lines confirmed to be silenced for Gal83 were also planted to ensure that the trait potential provided by this modification could be fully assessed. All planted tubers produced sprouts that emerged from the soil and developed into mature plants in the same way as controls. Tuber yields and number of tubers/plant were similar to those of the empty vector controls (Table 3). Thus, StGal83 gene silencing does not increase tuber quantities in the field.

Another way to silence StGal83 was to use the StGal83 promoter fragment (SEQ ID NO:6′) as an inverted repeat between AGP and GBSS promoters (pSIM1456). Primary tests in the greenhouse showed increased tuber set in line 1 (Table 4). A repeat experiment of pSIM1456 lines 1, 2, 12, 19 and 23 in the greenhouse showed a possible correlation between weak silencing of StGal83 and increase in tuber numbers. However, the promoter silencing of StGal83 did not increase tuber set in field (Table 5).

### Example 2

### PhH Gene Silencing does not Increase Tuber Quantity in the Field

Antisense inhibition of the cytosolic phosphorylase (PhH) gene had been suggested to increase the number of tubers produced per plant of the variety “Desiree” by 1.6 to 2.4-fold (Duwenig et al., Plant J 12: 323-333, 1997). In an attempt to confirm these data, “Bintje” was transformed with a transfer DNA carrying a silencing cassette designed to target the PhH gene (pSIM705). This cassette comprised two 499-bp fragments of the PhH gene (see SEQ ID NO:7 for cDNA, cDNA 8 for fragment), inserted as an inverted repeat between the 35S promoter of cauliflower mosaic virus and the terminator of the Ubi3 gene (SEQ ID NO:5), with the PAT intron (SEQ ID NO:9) between the inverted repeats of the PhH fragments. The transfer DNA also contained a selectable marker gene for kanamycin resistance. A total of 25 transgenic plants were propagated to produce lines, and three plants of each line were grown in the greenhouse together with both untransformed controls and transgenic controls carrying only the selectable marker gene. Data summarized in Table 6 demonstrates that PhH gene silencing did not correlate with an increase in tubers produced per plant. Similar results were obtained from a field trial (Table 7). Initial results suggested some increased tuber quantities in the field with an alternative silencing cassette (pSIM846) containing the PhH trailer (SEQ ID NO:10) between the tuber-specific promoters of the ADP glucose pyrophosphorylase gene (AGP) and the granule-bound starch synthase gene (GBSS) (SEQ ID NO:11) rather than the constitutive 35S promoter (Table 8). The PhH trailer was inserted as an inverted repeat isolated by the GBSS intron (SEQ ID NO:12). However, these results were not confirmed in a second-year field trial (Table 9). PhH gene silencing (pSIM705) did not alter tuber yield or set in the greenhouse or field.

### Example 3

### Overexpression of the StBel5 Gene does not Increase Tuber Quantity for Plants Grown in the Field

Overexpression of the StBel5 gene (SEQ ID NO:13) was reported to uncouple tuber set from day length in the SD plant species Solanum andigena when grown in the greenhouse. In contrast, it was found that transgenic “Bintje” potato plants containing this gene operably linked to the tuber-enhanced AGP promoter (pSIM1248) produced fewer but heavier potatoes than controls in the greenhouse (Table 10). Plants were propagated and five copies of each of five lines (1248-1, 3, 11, 15, 24) were planted in the field in Canyon County, Id., in May 2008. The number of tubers harvested from these lines showed that StBel5 did not increase tuber set. However, there was a trend towards reduced weights. A repeat of the field trial with 15 lines in 2011 showed the same results (Table 11).

### Example 4

### Ga20Ox1 Gene Silencing does not Increase Tuber Quantity in the Field

Reductions in GA20-oxidase 1 (Ga20ox1) gene expression had been indicated to double tuber numbers for Solanum tuberosum ssp. Andigena in growth chambers (Carrera et al., Plant J 22: 247-256, 2000). This phenotype was correlated with a substantial 37-58% reduction in stem height. The efficacy of this method was tested in “Bintje” by transforming plants with a construct containing both a selectable marker gene and a silencing cassette comprising two fragments of the Ga20ox1 gene (SEQ ID NO:14 for cDNA, SEQ ID NO:15 for fragment) inserted as inverted repeats between the 35S promoter and the Ubi3 terminator (pSIM703). Unlike the earlier report, this genetic modification was found to lower tuber yield in the greenhouse (Table 12). These reduced yields were not associated with increased tuber numbers. Only one line (pSIM703-50) produced more, smaller, tubers than controls in the greenhouse (Table 13). However, this construct was never tested in the field. An alternative construct (pSIM701) with the 35S promoter replaced by the Ubi3 promoter (SEQ ID NO:20) and 3′ end of GA20ox1 (SEQ ID NO:16) also generated only one line (pSIM701-69) with apparently reduced tuber size in the greenhouse (Tables 14a, b). Subsequent field trials did not confirm this result (Table 15).

Silencing both GA20ox1 and Ga20ox2 with trailers (SEQ ID NO:17 for StGA20ox trailer, SEQ ID NO:18 for StGA20ox1, and SEQ ID NO:19 for StGA20ox2 trailer) as inverted repeats under control of Ubi3 promoter (SEQ ID NO:19) still did not increase tuber number (Table 16).

### Example 5

### Increased Carotenoid Formation does not Increase Tuber Quantity in the Field

In an attempt to increase carotenoid content of tubers, both the phytoene synthase (Psy) gene from maize (SEQ ID NO:22) and the phytoene desaturase (CrtI) gene from chimeric bacterial Erwinia spp. (SEQ ID NO:23) were overexpressed, driven by the tuber-specific promoters AGP and GBSS, respectively (pSIM1457). A second construct was made which overexpressed the Solanum lycopersicum chromoplast-specific lycopene beta-cyclase (LeLcyB, see SEQ ID NO:24, pSIM1469). Later it was found that LeLcyB showed 99% homology with tomato neoxathin synthase (LeNXS), and only 50% with LeLcyB. In an attempt to further increase carotenoids, the two constructs were combined, which led not only to an increase in carotenoid content, but also consistently produced an increased number of tubers in the greenhouse as compared to pSIM1457 only (Table 18).

Selected lines were again grown in the greenhouse, using now 2-gallon pots, and the tuber number increase and size reduction were confirmed (Table 19). Unfortunately, the tuber set increase in the field was not as high as in the greenhouse, and yields were highly reduced.

### Example 6

### Overexpression of NXS, Together with Silencing of StCYP, StChxE and StZep Increased the Number of Tubers Produced Per Plant in the Field

To identify genes associated with increased tuber numbers, “Bintje” was transformed with three pools of Agrobacterium strains. The transformation vectors carried, in addition to pool-specific selection markers, expression cassettes for at least 1 of 23 different plant genes involved in the biosynthesis or metabolism of carotenoids. Selection of transformed cells for resistance against three selection agents yielded 1,683 transgenic shoots. These shoots were allowed to root, planted in soil and transferred to the greenhouse. Tubers were harvested after three months. Line BB3-6 showed a 2.4× increase in tuber numbers over the control (Table 20).

Molecular analysis found that line BB3-6 contained constructs pSIM1469 and pSIM1891. The construct pSIM1469 contains a LeNXS gene (SEQ ID NO:24) over-expression cassette with AGP promoter; the pSIM1891 contains a silencing cassette for cytochrome P450-type monooxygenase (StCYP, see SEQ ID NO:25 for the cDNA and SEQ ID NO:26 and SEQ 27 for the fragments used in silencing), and zeaxanthin epoxidase (StZep, see SEQ ID 30 for the cDNA and SEQ ID NO:31 for the silencing fragments).

### Example 7

### Silencing StGA20ox1 with TRUNCATED UBI7s and Ubi3 Promoter

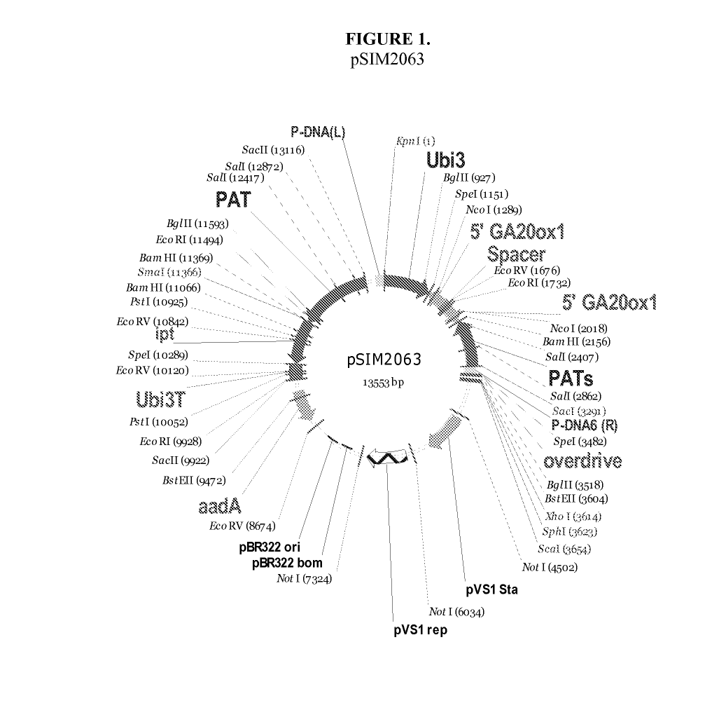

A construct was made to silence StGA20ox1 with TRUNCATED UBI7s and Ubi3 promoter (pSIM2063, FIG. 1) (See SEQ ID NO: 15 for GA20ox1 silencing fragment, SEQ ID NO: 33 for spacer between GA20ox1 invert repeat). Marker-free, all-native DNA transformation was carried out as described before (Richael et al., 2008). No potato lines transformed with the T-DNA of this construct produced more tubers per plant than the untransformed controls when grown in the field. SEQ IDs for the various parts of the silencing cassette of pSIM2064 are shown, from 5′ to 3′, as nrs. 36 (Ubi3 promoter), 37 (StGa21ox1 fragment in antisense orientation), 38 (spacer), 39 (StGa20ox1 fragment in sense orientation), and 40 (Truncated Ubi7 promoter in inverse orientation).

### Example 8

### Silencing StCYP, StChxE and StZep and Over-Expression of Modified StNXS

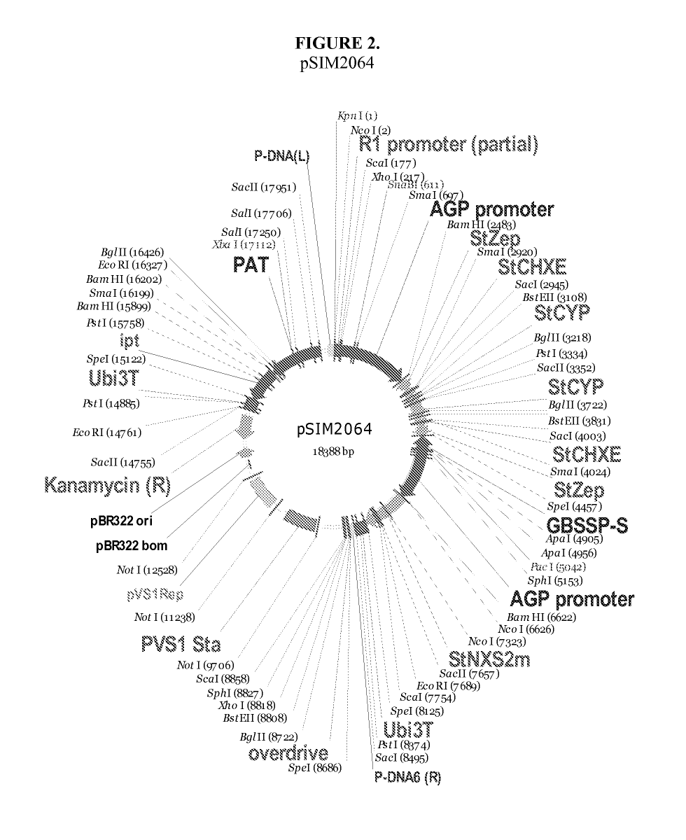

The construct pSIM2064 (FIG. 2) contains two expression cassettes: (1) silencing cassette of StCYP and StZep, which is the same as in pSIM1891, except that the Ubi3 terminator was replaced with the Ubi3 promoter; (2) Over-expression of modified StNXS with AGP promoter (See SEQ ID NO:34 for StNXSm cDNA sequence). A partial sequence of R1 promoter (SEQ ID NO:35) was inverted between LB and silencing cassette as a spacer. Marker-free, all-native DNA transformation was carried out as described before (Richael et al., 2008). Some potato lines transformed with the T-DNA of this construct produce more tubers per plant than the untransformed controls when grown in the field. SEQ IDs for the various parts of the silencing cassette of pSIM2064 are shown, from 5′ to 3′, as SEQ ID NOs: 41 (partial R1 promoter, used as spacer upstream from the Agp promoter), 42 (Agp promoter), 43 (antisense fragment of StZep), 45 (antisense fragment of StCyp), 46 (sense fragment of StCyp), 48 (sense fragment of StZep), and 49 (Gbss promoter in inverse orientation). The additional overexpression cassette consists of, from 5′ to 3′, Agp promoter (SEQ ID NO:50), StNxs gene (SEQ ID NO:51), and Ubi3 terminator (SEQ ID NO:52).

### Example 9

### 4-5 Fold Increased Tuber Set in Line IO7-11G

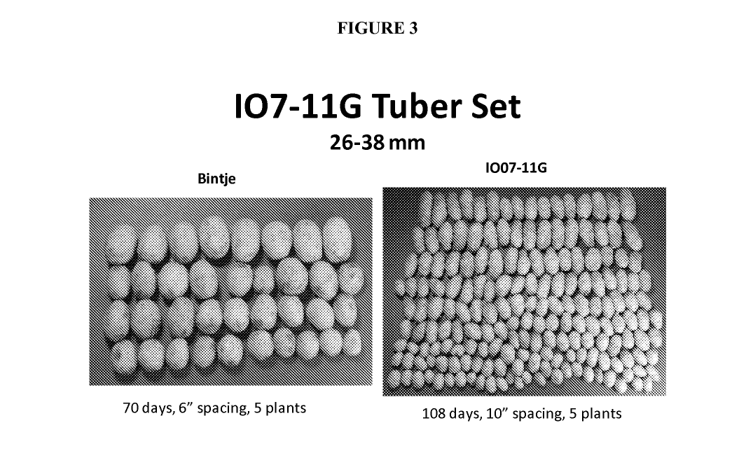

In an effort to lower grower costs and increase the sustainability of producing potatoes, we transformed the potato variety “Bintje” with three pools of Agrobacterium strains, each of which contained an expression cassette designed to increase or reduce the expression of one or several gene(s) predicted to be involved in tuber set. The Agrobacterium pools contain 52 different binary vectors. About 1800 regenerated events were transferred to the greenhouse, allowed to mature, and analyzed for tuber set. The lines with increased tuber set were characterized molecularly to understand which modifications provide the best results. Line IO7-11G was confirmed to increased tuber set 4-5 fold in the field trials (Table 22, FIG. 3). For BabyBaker (26-38 mm), IO7-11G increase tuber 15 times compared to Bintje wild type.

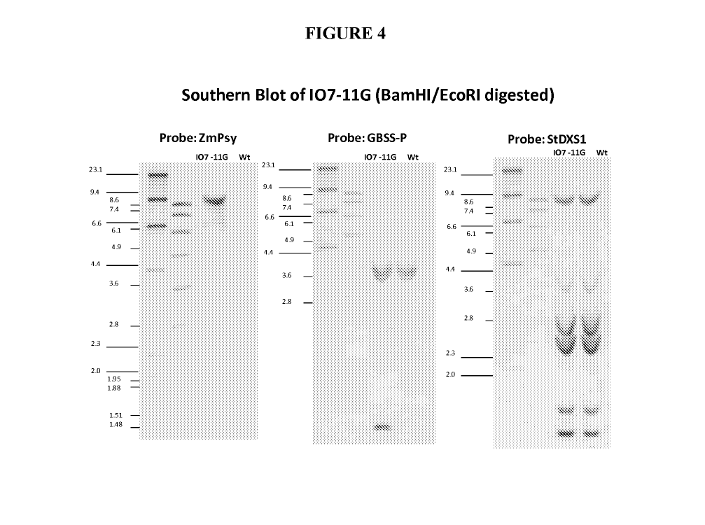

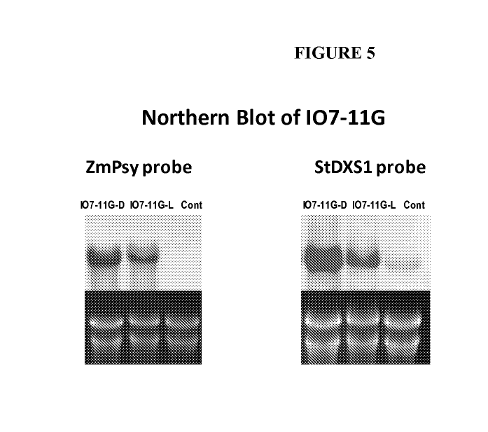

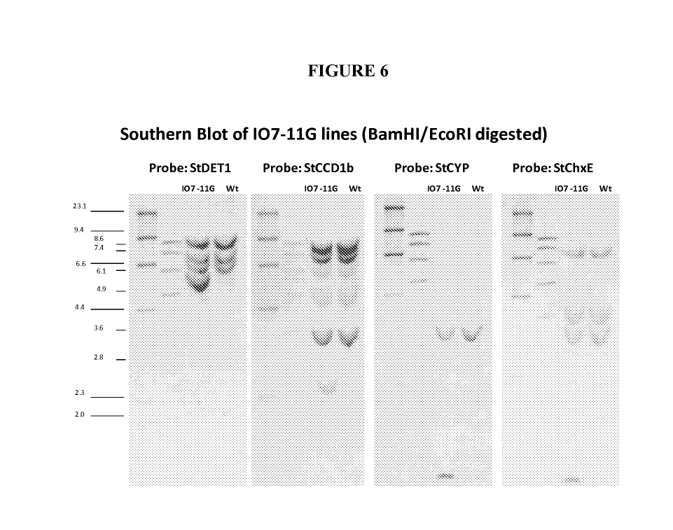

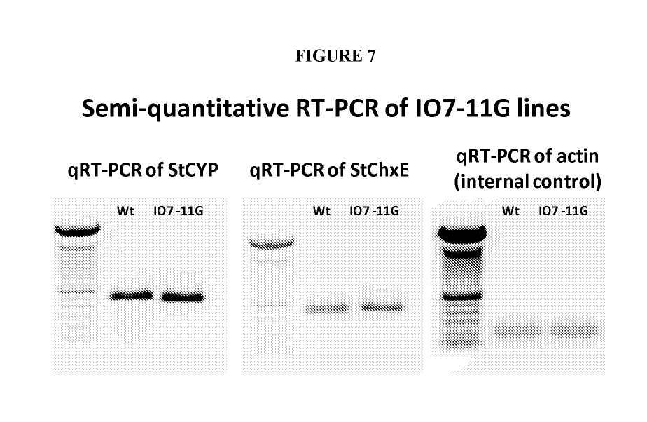

Primary PCR showed that tubers of line IO7-11G over-expressed ZmPsy (SEQ ID 22) but displayed down-regulated expression levels for DET1 (SEQ ID 53-55), CCD 1b (SEQ ID 56-58) and CYP (SEQ ID 25-27). Southern blot data confirmed the line contained the ZmPsy gene operably linked to the GBSS promoter (FIG. 4). Northern blot analysis confirmed increased expression of ZmPsy (FIG. 5). Interestingly, tubers stored in the dark, accumulated higher ZmPsy transcript levels than when exposed to light. This phenomenon also applied to a second gene, StDXS 1, that was not present in IO7-11G as transgene and appeared to be induced indirectly (FIG. 5, FIG. 4). Southern blot of silenced genes showed there are DET1, CCD 1b and CYP cassettes in line IO7-11G (FIG. 6). However, the CYP gene was truncated (FIG. 6) and semi-quantitative RT-PCR showed no reduction of CYP expression (FIG. 7).

### TABLES

TABLE 1
StGal83 gene silencing (1448) in the greenhouse (1-gallon pots).
“401” lines represent transgenic controls.

| Line # | Avg Tuber # | StDev |
|---|---|---|
| 401-1 (C) | 20.3 | 4.0 |
| 401-2 (C) | 18.3 | 4.2 |
| 401-4 (C) | 10.0 | 2.6 |
| 401-5 (C) | 19.3 | 5.9 |
| 401-6 (C) | 12.3 | 3.1 |
| Bintje | 8.0 | 3.0 |
| 1448-1 | 10.3 | 4.2 |
| 1448-2 | 8.7 | 2.3 |
| 1448-3 | 12.0 | 6.1 |
| 1448-4 | 10.3 | 4.2 |
| 1448-5 | 11.0 | 5.3 |
| 1448-6 | 7.3 | 2.3 |
| 1448-7 | 9.0 | 2.6 |
| 1448-8 | 12.7 | 3.1 |
| 1448-9 | 9.7 | 1.5 |
| 1448-10 | 13.7 | 6.7 |
| 1448-11 | 6.0 | 1.0 |
| 1448-12 | 13.7 | 2.5 |
| 1448-13 | 19.3 | 5.0 |
| 1448-14 | 12.3 | 1.5 |
| 1448-15 | 18.3 | 3.1 |
| 1448-16 | 2.7 | 0.6 |
| 1448-17 | 8.0 | 2.0 |
| 1448-18 | 6.0 | 1.0 |
| 1448-19 | 11.7 | 2.5 |
| 1448-20 | 5.0 | 1.0 |
| 1448-21 | 20.3 | 6.7 |
| 1448-22 | 13.7 | 2.9 |
| 1448-23 | 12.0 | 4.6 |
| 1448-24 | 17.3 | 5.5 |
| 1448-25 | 12.7 | 0.6 |

TABLE 2
StGal83 gene silencing (1448-13, 15, 21, 24) in the greenhouse
(2-gallon pots). “401” lines represent transgenic controls.

| Line | Avg Tuber # | StDev |
|---|---|---|
| Bintje | 20.0 | 4.5 |
| 401-1 | 15.3 | 5.1 |
| 401-2 | 18.7 | 5.1 |
| 401-5 | 18.0 | 1.0 |
| 846-1 | 32.7 | 3.1 |
| 1448-13 | 32.7 | 4.0 |
| 1448-15 | 16.3 | 0.5 |
| 1448-21 | 11.3 | 0.5 |
| 1448-24 | 17.0 | 2.8 |

TABLE 3
StGal83 gene silencing (1448) in the field. “401” lines
represent transgenic controls.

| Line | Tuber # |
|---|---|
| 401-1 | 52 |
| 401-2 | 75 |
| 401-3 | 66 |
| 401-4 | 39 |
| 401-5 | 46 |
| 401-6 | 63 |
| 401-7 | 49 |
| 401-8 | 58 |
| 401-9 | 52 |
| 401-10 | 38 |
| 401-11 | 62 |
| 401-12 | 76 |
| 401-13 | 73 |
| 401-14 | 77 |
| 401-15 | 54 |
| Bintje-1 | 46 |
| Bintje-2 | 45 |
| Bintje-3 | 53 |
| Bintje-4 | 56 |
| Bintje-5 | 45 |
| 1448-1 | 26 |
| 1448-2 | 30 |
| 1448-3 | 42 |
| 1448-4 | 46 |
| 1448-5 | 38 |
| 1448-6 | 48 |
| 1448-7 | 44 |
| 1448-8 | 70 |
| 1448-9 | 52 |
| 1448-10 | 30 |
| 1448-11 | 45 |
| 1448-12 | 51 |
| 1448-13 | 41 |
| 1448-14 | 62 |
| 1448-15 | 26 |

TABLE 4
StGal83 Promoter Silencing (1456) in the greenhouse. “401” lines
represent transgenic controls.

| Line | Avg Tuber # | StDev |
|---|---|---|
| 401-1 | 20.3 | 4.0 |
| 401-2 | 18.3 | 4.0 |
| 401-4 | 10.0 | 2.6 |
| 401-5 | 19.3 | 5.9 |
| 401-6 | 12.3 | 3.1 |
| Bintje | 8.0 | 3.0 |
| 1456-1 | 39.7 | 9.5 |
| 1456-2 | 15.3 | 8.7 |
| 1456-3 | 8.7 | 1.5 |
| 1456-4 | 10.7 | 2.1 |
| 1456-5 | 13.0 | 3.6 |
| 1456-6 | 10.3 | 1.5 |
| 1456-7 | 13.3 | 2.1 |
| 1456-8 | 7.0 | 2.0 |
| 1456-9 | 11.0 | 2.6 |
| 1456-10 | 9.0 | 2.0 |
| 1456-11 | 7.0 | 1.0 |
| 1456-12 | 14.0 | 4.4 |
| 1456-13 | 6.3 | 0.6 |
| 1456-14 | 11.0 | 2.0 |
| 1456-15 | 4.7 | 2.1 |
| 1456-16 | 10.3 | 1.2 |
| 1456-17 | 10.0 | 4.4 |
| 1456-18 | 12.0 | 1.7 |
| 1456-19 | 15.0 | 5.3 |
| 1456-20 | 9.0 | 1.0 |
| 1456-21 | 11.3 | 1.5 |
| 1456-22 | 11.7 | 3.1 |
| 1456-23 | 15.0 | 7.2 |
| 1456-24 | 9.7 | 5.5 |
| 1456-25 | 10.0 | 4.4 |

TABLE 5
StGal83 promoter silencing (1456) in the field. “401” lines
represent transgenic controls.

| Line | Tuber # |
|---|---|
| 401-1 | 52 |
| 401-2 | 75 |
| 401-3 | 66 |
| 401-4 | 39 |
| 401-5 | 46 |
| 401-6 | 63 |
| 401-7 | 49 |
| 401-8 | 58 |
| 401-9 | 52 |
| 401-10 | 38 |
| 401-11 | 62 |
| 401-12 | 76 |
| 401-13 | 73 |
| 401-14 | 77 |
| 401-15 | 54 |
| Bintje-1 | 46 |
| Bintje-2 | 45 |
| Bintje-3 | 53 |
| Bintje-4 | 56 |
| Bintje-5 | 45 |
| 1456-1 | 30 |
| 1456-2 | 46 |
| 1456-3 | 45 |
| 1456-4 | 43 |
| 1456-5 | 53 |
| 1456-6 | 56 |
| 1456-7 | 54 |
| 1456-8 | 61 |
| 1456-9 | 54 |
| 1456-10 | 39 |
| 1456-11 | 37 |
| 1456-12 | 15 |
| 1456-13 | 56 |
| 1456-14 | 50 |
| 1456-15 | 41 |

TABLE 6
PhH gene silencing (705) in the greenhouse. “401” lines
represent transgenic controls.

| | Avg | |
|---|---|---|
| Line | Tuber # | StDev |
| 401-1 | 11.0 | 2.6 |
| 401-2 | 11.0 | 1.7 |
| 401-4 | 6.0 | 3.5 |
| 401-5 | 13.0 | 1.0 |
| 401-6 | 11.7 | 2.3 |
| 401-8 | 12.3 | 4.0 |
| 401-9 | 13.3 | 5.5 |
| 401-11 | 12.0 | 0.0 |
| 401-13 | 12.3 | 2.3 |
| 401-14 | 10.7 | 3.1 |
| Bintje | 9.2 | 2.3 |
| 705-11 | 17.7 | 3.2 |
| 705-20 | 12.3 | 3.2 |
| 705-21 | 16.0 | 7.0 |
| 705-26 | 20.3 | 5.1 |
| 705-27 | 7.0 | 1.0 |
| 705-28 | 9.7 | 2.1 |
| 705-30 | 12.3 | 3.8 |
| 705-32 | 10.0 | 5.3 |
| 705-34 | 11.7 | 2.5 |
| 705-35 | 12.7 | 2.3 |
| 705-36 | 14.0 | 0.0 |
| 705-37 | 15.5 | 0.7 |
| 705-39 | 11.0 | 4.6 |
| 705-41 | 12.0 | 4.4 |
| 705-43 | 7.3 | 4.5 |
| 705-45 | 9.3 | 3.8 |
| 705-46 | 17.3 | 5.5 |
| 705-47 | 17.3 | 4.7 |
| 705-49 | 8.0 | 1.0 |
| 705-51 | 11.0 | 3.6 |
| 705-52 | 10.0 | 1.7 |
| 705-54 | 9.3 | 1.5 |
| 705-55 | 8.3 | 2.1 |
| 705-56 | 10.3 | 2.1 |
| 705-57 | 7.7 | 0.6 |

TABLE 7
PhH gene silencing (705) in the field. “401” lines represent
transgenic controls.

| Line | Tuber # |
|---|---|
| 401-1 | 52 |
| 401-2 | 75 |
| 401-3 | 66 |
| 401-4 | 39 |
| 401-5 | 46 |
| 401-6 | 63 |
| 401-7 | 49 |
| 401-8 | 58 |
| 401-9 | 52 |
| 401-10 | 38 |
| 401-11 | 62 |
| 401-12 | 76 |
| 401-13 | 73 |
| 401-14 | 77 |
| 401-15 | 54 |
| Bintje-1 | 46 |
| Bintje-2 | 45 |
| Bintje-3 | 53 |
| Bintje-4 | 56 |
| Bintje-5 | 45 |
| 705-1 | 7 |
| 705-2 | 36 |
| 705-3 | 40 |
| 705-4 | 48 |
| 705-5 | 54 |
| 705-6 | 86 |
| 705-7 | 32 |
| 705-8 | 76 |
| 705-9 | 31 |
| 705-10 | 50 |
| 705-11 | 10 |
| 705-12 | 11 |
| 705-13 | 83 |
| 705-14 | 26 |
| 705-15 | 61 |

TABLE 8
PhH gene silencing with ADP and GBSS promoters (846) in the
greenhouse. “401” lines represent transgenic controls.

| | Avg | |
|---|---|---|
| Line | Tuber # | StDev |
| 401-1 | 19.3 | 3.8 |
| 401-2 | 21.7 | 5.9 |
| 401-6 | 17.7 | 8.0 |
| 401-8 | 20.7 | 2.1 |
| Bintje | 19.8 | 2.5 |
| 846-1 | 32.7 | 4.0 |
| 846-2 | 16.3 | 0.5 |
| 846-3 | 11.3 | 0.5 |
| 846-4 | 17.0 | 2.8 |
| 846-5 | 13.7 | 0.9 |
| 846-7 | 14.7 | 0.9 |
| 846-9 | 13.0 | 2.2 |
| 846-11 | 18.0 | 3.6 |
| 846-12 | 15.7 | 2.1 |
| 846-13 | 17.3 | 0.5 |
| 846-15 | 15.3 | 0.5 |
| 846-17 | 21.3 | 5.8 |
| 846-18 | 17.7 | 7.4 |
| 846-20 | 10.7 | 2.5 |
| 846-21 | 15.3 | 1.9 |
| 846-22 | 24.3 | 3.3 |
| 846-24 | 13.3 | 2.1 |
| 846-25 | 13.7 | 4.5 |
| 846-26 | 14.7 | 0.9 |
| 846-28 | 12.7 | 5.4 |
| 846-29 | 13.3 | 0.9 |
| 846-30 | 18.5 | 5.5 |
| 846-31 | 10.0 | 0.0 |
| 846-32 | 16.7 | 2.9 |
| 846-33 | 10.3 | 4.1 |

TABLE 9
PhH gene silencing with ADP and GBSS promoters (846) in the field.
“401” lines represent transgenic controls.

| Line | Tuber # |
|---|---|
| 401-1 | 52 |
| 401-2 | 75 |
| 401-3 | 66 |
| 401-4 | 39 |
| 401-5 | 46 |
| 401-6 | 63 |
| 401-7 | 49 |
| 401-8 | 58 |
| 401-9 | 52 |
| 401-10 | 38 |
| 401-11 | 62 |
| 401-12 | 76 |
| 401-13 | 73 |
| 401-14 | 77 |
| 401-15 | 54 |
| Bintje-1 | 46 |
| Bintje-2 | 45 |
| Bintje-3 | 53 |
| Bintje-4 | 56 |
| Bintje-5 | 45 |
| 846-1 | 49 |
| 846-2 | 57 |
| 846-3 | 78 |
| 846-4 | 27 |
| 846-5 | 66 |
| 846-6 | 89 |
| 846-7 | 34 |
| 846-8 | 59 |
| 846-9 | 39 |
| 846-10 | 35 |
| 846-11 | 68 |
| 846-12 | 23 |
| 846-13 | 20 |
| 846-14 | 47 |
| 846-15 | 75 |

TABLE 10
StBel5 gene overexpression (1248) in the greenhouse. “401” lines
represent transgenic controls.

| | Avg | | Avg | |
|---|---|---|---|---|
| Line | Tuber # | StDev | Weight (g) | StDev |
| 401-1 | 15.7 | 6.4 | 472.3 | 112.5 |
| 401-2 | 18.7 | 4.9 | 457.0 | 69.5 |
| 401-6 | 6.7 | 2.5 | 170.0 | 65.7 |
| Bintje | 13.7 | 4.2 | 483.7 | 48.4 |
| 1248-1 | 7.7 | 1.2 | 531.0 | 37.7 |
| 1248-2 | 10.3 | 3.3 | 483.3 | 68.2 |
| 1248-3 | 8.0 | 0.8 | 532.3 | 48.3 |
| 1248-4 | 11.3 | 0.9 | 495.0 | 7.8 |
| 1248-5 | 3.0 | 0.0 | 28.5 | 3.5 |
| 1248-6 | 10.7 | 1.2 | 511.0 | 49.5 |
| 1248-7 | 11.0 | 2.2 | 490.3 | 33.9 |
| 1248-8 | 11.0 | 2.2 | 505.3 | 40.1 |
| 1248-9 | 14.0 | 0.8 | 528.3 | 15.2 |
| 1248-10 | 15.7 | 2.4 | 539.3 | 5.3 |
| 1248-11 | 8.0 | 0.8 | 503.0 | 54.5 |
| 1248-12 | 7.5 | 1.5 | 377.0 | 129.0 |
| 1248-13 | 6.7 | 2.1 | 96.7 | 46.6 |
| 1248-14 | 3.0 | 0.8 | 18.3 | 1.9 |
| 1248-15 | 8.3 | 1.7 | 444.3 | 46.7 |
| 1248-16 | 11.3 | 4.0 | 533.0 | 64.5 |
| 1248-17 | 7.3 | 0.5 | 431.7 | 124.6 |
| 1248-18 | 12.0 | 2.9 | 519.0 | 6.5 |
| 1248-19 | 16.7 | 4.5 | 544.7 | 27.1 |
| 1248-20 | 11.0 | 2.2 | 550.7 | 6.1 |
| 1248-21 | 9.0 | 1.4 | 492.7 | 52.3 |
| 1248-22 | 11.3 | 1.7 | 504.3 | 20.9 |
| 1248-23 | 8.0 | 2.2 | 496.3 | 29.8 |
| 1248-24 | 5.7 | 1.6 | 433.3 | 55.4 |
| 1248-25 | 9.0 | 0.0 | 520.7 | 82.6 |

TABLE 11
StBel5 gene overexpression (1248) in the field. “401” lines
represent transgenic controls.

| Line | Tuber # |
|---|---|
| 401-1 | 52 |
| 401-2 | 75 |
| 401-3 | 66 |
| 401-4 | 39 |
| 401-5 | 46 |
| 401-6 | 63 |
| 401-7 | 49 |
| 401-8 | 58 |
| 401-9 | 52 |
| 401-10 | 38 |
| 401-11 | 62 |
| 401-12 | 76 |
| 401-13 | 73 |
| 401-14 | 77 |
| 401-15 | 54 |
| Bintje-1 | 46 |
| Bintje-2 | 45 |
| Bintje-3 | 53 |
| Bintje-4 | 56 |
| Bintje-5 | 45 |
| 1248-1 | 28 |
| 1248-2 | 35 |
| 1248-3 | 42 |
| 1248-4 | 46 |
| 1248-5 | 39 |
| 1248-6 | 54 |
| 1248-7 | 45 |
| 1248-8 | 9 |
| 1248-9 | 46 |
| 1248-10 | 39 |
| 1248-11 | 40 |
| 1248-12 | 46 |
| 1248-13 | 38 |
| 1248-14 | 53 |
| 1248-15 | 30 |

TABLE 12
Ga20ox1 gene silencing (pSIM703) in the greenhouse. “401” lines
represent transgenic controls.

| | Avg | |
|---|---|---|
| Line | Tuber # | StDev |
| 401-1 | 2.6 | 0.2 |
| 401-2 | 2.4 | 0.4 |
| 401-4 | 1.7 | 0.1 |
| 401-5 | 2.5 | 0 |
| 401-6 | 3.1 | 0.3 |
| 401-8 | 2.3 | 0.7 |
| 401-9 | 2.5 | 0.2 |
| 401-11 | 2.5 | 0.3 |
| 401-13 | 2.3 | 0.3 |
| 401-14 | 2.5 | 0.3 |
| Bintje | 2.4 | 0.3 |
| 703-32 | 0.6 | 0.1 |
| 703-36 | 0.8 | 0.1 |
| 703-37 | 1.1 | 0.3 |
| 703-39 | 1.2 | 0.4 |
| 703-40 | 1.3 | 0.1 |
| 703-41 | 1.5 | 0.2 |
| 703-42 | 1.2 | 0.2 |
| 703-45 | 1.2 | 0.3 |
| 703-50 | 1.4 | 0.1 |
| 703-51 | 1.5 | 0.2 |
| 703-52 | 1.5 | 0.1 |
| 703-54 | 1.2 | 0.2 |
| 703-55 | 1.2 | 0.2 |
| 703-58 | 1.2 | 0.03 |
| 703-59 | 1.3 | 0.2 |
| 703-60 | 1.6 | 0.2 |
| 703-61 | 2.3 | 0.1 |
| 703-65 | 1.3 | 0.3 |
| 703-66 | 1.2 | 0.1 |
| 703-67 | 1.4 | 0.2 |
| 703-71 | 1.1 | 0.1 |
| 703-73 | 2.1 | 0.3 |
| 703-74 | 1.4 | 0.2 |
| 703-76 | 1.2 | 0.1 |
| 703-77 | 1.5 | 0.1 |

TABLE 13
Ga20ox1gene silencing (pSIM703-50) versus control in greenhouse.
“401” lines represent transgenic controls.

| | 401- | 703- |
|---|---|---|
| Size | 99 | 50 |
| <1.5 | 1.0 | 7.0 |
| 1.5-3 | 15.0 | 28.0 |
| 3-4.5 | 12.0 | 12.0 |
| 4.5-6 | 10.0 | 10.0 |
| 6-7.5 | 2.0 | 1.0 |
| >7.5 | 0.0 | 0.0 |
| Total | 40.0 | 58.0 |

TABLE 14
Ga20ox1 gene silencing (with alternative construct pSIM701) in
greenhouse.

| | Avg | |
|---|---|---|
| Line | Tuber # | StDev |
| 401-1 | 11.0 | 2.6 |
| 401-2 | 11.0 | 1.7 |
| 401-4 | 6.0 | 3.5 |
| 401-5 | 13.0 | 1.0 |
| 401-6 | 11.7 | 2.3 |
| 401-8 | 12.3 | 4.0 |
| 401-9 | 13.3 | 5.5 |
| 401-11 | 12.0 | 0.0 |
| 401-13 | 12.3 | 2.3 |
| 401-14 | 10.7 | 3.1 |
| Bintje | 9.2 | 2.3 |
| 701-34 | 14.3 | 5.0 |
| 701-37 | 8.7 | 2.1 |
| 701-39 | 7.7 | 0.6 |
| 701-44 | 15.0 | 5.2 |
| 701-46 | 13.0 | 1.7 |
| 701-51 | 11.0 | 1.0 |
| 701-52 | 11.0 | 3.0 |
| 701-53 | 13.7 | 4.0 |
| 701-54 | 8.3 | 3.8 |
| 701-55 | 9.7 | 2.1 |
| 701-56 | 10.7 | 1.2 |
| 701-57 | 17.0 | 1.0 |
| 701-58 | 12.3 | 4.0 |
| 701-59 | 8.3 | 4.9 |
| 701-61 | 18.3 | 3.2 |
| 701-62 | 17.3 | 3.5 |
| 701-65 | 9.3 | 2.5 |
| 701-66 | 14.0 | 2.6 |
| 701-67 | 16.0 | 4.0 |
| 701-68 | 7.0 | 4.4 |
| 701-69 | 16.7 | 2.9 |
| 701-71 | 10.3 | 2.1 |
| 701-74 | 11.7 | 1.5 |
| 701-75 | 13.0 | 2.6 |
| 701-76 | 7.3 | 2.5 |

TABLE 14B
pSIM701-69 (Ga20ox1 gene silencing, alternative construct) versus
control in greenhouse.

| Size | 401-99 | 701-69 |
|---|---|---|
| <1.5 | 1.0 | 12 |
| 1.5-3 | 15.0 | 19 |
| 3-4.5 | 12.0 | 18 |
| 4.5-6 | 10.0 | 0 |
| 6-7.5 | 2.0 | 1 |
| >7.5 | 0.0 | 0 |
| Total | 40.0 | 50.0 |

TABLE 15
Ga20ox1 gene silencing (alternative construct pSIM701) in field.

| Line | Avg | StDev | StError |
|---|---|---|---|
| 401-5 | 211.5 | 20.5 | 5.1 |
| 401-6 | 166.5 | 4.5 | 1.1 |
| 401-9 | 215.5 | 6.5 | 1.6 |
| 401-11 | 163.5 | 15.5 | 3.9 |
| 401-14 | 178.0 | 4.0 | 1.0 |
| Bintje | 174.0 | 11.0 | 2.8 |
| 701-34 | 123.0 | 15.0 | 3.8 |
| 701-44 | 118.0 | 1.0 | 0.3 |
| 701-53 | 103.0 | 5.0 | 1.3 |
| 701-57 | 74.0 | 16.0 | 4.0 |
| 701-58 | 76.0 | 14.0 | 3.5 |
| 701-61 | 129.0 | 29.0 | 7.3 |
| 701-62 | 103.0 | 14.0 | 3.5 |
| 701-66 | 110.5 | 15.5 | 3.9 |
| 701-67 | 99.0 | 13.0 | 3.3 |
| 701-69 | 85.5 | 14.5 | 3.6 |

TABLE 16
Ga20ox1 and Ga20ox2 gene silencing (pSIM262) in the field.

| Line | Tuber # |
|---|---|
| 401-1 | 52 |
| 401-2 | 75 |
| 401-3 | 66 |
| 401-4 | 39 |
| 401-5 | 46 |
| 401-6 | 63 |
| 401-7 | 49 |
| 401-8 | 58 |
| 401-9 | 52 |
| 401-10 | 38 |
| 401-11 | 62 |
| 401-12 | 76 |
| 401-13 | 73 |
| 401-14 | 77 |
| 401-15 | 54 |
| Bintje-1 | 46 |
| Bintje-2 | 45 |
| Bintje-3 | 53 |
| Bintje-4 | 56 |
| Bintje-5 | 45 |
| 262-1 | 58 |
| 262-2 | 74 |
| 262-3 | 41 |
| 262-4 | 33 |
| 262-5 | 78 |
| 262-6 | 38 |
| 262-7 | 76 |
| 262-8 | 42 |
| 262-9 | 72 |
| 262-10 | 84 |
| 262-11 | 61 |
| 262-12 | 73 |
| 262-13 | 68 |
| 262-14 | 47 |
| 262-15 | 19 |

TABLE 17
Psy, Crtl and LeLcyB overexpression (pSIM1457 in 1469) in the
greenhouse.

| Line | Tuber # |
|---|---|
| Bintje | 6 |
| Bintje | 9 |
| Bintje | 9 |
| Bintje | 11 |
| Bintje | 9 |
| 1457-11 | 3 |
| 1457-11 | 10 |
| 1457-11 | 9 |
| 1469-1 | 10 |
| 1469-2 | 46 |
| 1469-3 | 11 |
| 1469-4 | 14 |
| 1469-5 | 15 |
| 1469-6 | 10 |
| 1469-7 | 21 |
| 1469-8 | 23 |
| 1469-9 | 27 |
| 1469-10 | 4 |
| 1469-11 | 6 |
| 1469-12 | 18 |
| 1469-13 | 47 |
| 1469-14 | 11 |
| 1469-15 | 26 |
| 1469-16 | 19 |
| 1469-17 | 12 |
| 1469-18 | 29 |
| 1469-19 | 12 |
| 1469-20 | 37 |
| 1469-21 | 10 |
| 1469-22 | 37 |
| 1469-23 | 8 |
| 1469-24 | 11 |
| 1469-25 | 15 |

TABLE 18
Repeat of Psy, Crtl and LeLcyB overexpression (pSIM1457 in 1469) in
2-gallon pots.

| Line | Avg | StDev |
|---|---|---|
| Bintje | 13 | 0 |
| 1457/1469-2 | 39 | 5.66 |
| 1457/1469-13 | 40 | 2.83 |
| 1457/1469-20 | 37 | 7.07 |
| 1457/1469-22 | 34.5 | 2.12 |

TABLE 19
Line BB3-6 in the field.

| Line | Avg | StDev |
|---|---|---|
| 401 | 58.7 | 12.6 |
| Bintje | 49 | 4.6 |
| BB3-6 | 129.4 | 11.6 |

TABLE 20
IO7-11G in field.

| | tuber # | | | total |
|---|---|---|---|---|
| line | undersize | 26-38 mm | oversize | tuber # |
| Bintje wt | 3 | 12 | 46 | 61 |
| IO7-11G | 57 | 185 | 0 | 242 |

What is claimed is:

1. A method for increasing tuber yield production in a potato plant comprising (A) overexpressing in a potato plant a neoxathin synthase gene and (B) downregulating in the same potato plant the expression of at least one of (i) cytochrome P450-type monooxygenase and (ii) zeaxanthin epoxidase, wherein the potato plant yields more mature tubers than a control potato plant.

2. The method of claim 1, wherein the potato plant is a variety selected from the group consisting of Bintje, Atlantic, Russet Burbank, Russet Ranger, Bondi and Moonlight.

3. The method of claim 1, wherein (i) cytochrome P450-type monooxygenase and (ii) zeaxanthin epoxidase are both downregulated in the potato plant.

4. A potato plant comprising in its genome an expression cassette for overexpressing a neoxathin synthase gene and at least one gene silencing expression cassette selected from the group consisting of (i) a gene silencing cassette for down-regulating cytochrome P450-type monooxygenase and (ii) a gene silencing cassette for down-regulating zeaxanthin epoxidase

5. The potato plant of claim 4, wherein the plant's genome comprises both gene silencing cassettes of (i) and (ii).

6. The potato plant of claim 5, wherein the potato plant has an increased tuber yield production compared to a wild potato plant of the same variety.

7. The potato plant of claim 6, wherein the plant produces mature tubers having an average size of 26 to 38 mm.

8. A heat-processed product of the potato plant of claim 7, wherein the heat-processed product has superior flavor, texture and appearance compared to a heat-processed product of a wild potato plant of the same variety.

9. The heat-processed product of claim 8, wherein the heat-processed product is a French fry or a roasted potato containing up to 30% of the oil content of a French fry or roasted potato of a wild potato plant of the same variety.

10. A vector comprising (A) an expression cassette for expressing a neoxathin synthase gene; and (B) at least one gene silencing expression cassette selected from the group consisting of (i) a gene silencing cassette for down-regulating cytochrome P450-type monooxygenase and (ii) a gene silencing cassette for down-regulating zeaxanthin epoxidase.

11. The vector of claim 10, wherein the vector comprises both gene silencing cassettes of (i) and (ii).

12. A method for increasing tuber yield production in a potato plant comprising over-expressing in a potato plant a phytoetene synthase gene and down-regulating in the same potato plant the expression of at least one of (i) de-etiolated homolog 1, (ii) carotenoid dioxygenase 1B and (iii) cytochrome P450-type monooxygenase, wherein the potato plant yields more mature tubers than a control potato plant.

13. The method of claim 12, wherein the potato plant is a variety selected from the group consisting of Bintje, Atlantic, Russet Burbank, Russet Ranger, Bondi and Moonlight.

14. The method of claim 12, wherein (i) de-etiolated homolog 1, (ii) carotenoid dioxygenase 1B and (iii) cytochrome P450-type monooxygenase are all down-regulated in the potato plant.

15. A potato plant comprising in its genome an expression cassette for over-expressing a phytoetene synthase gene and at least one gene silencing expression cassette selected from the group consisting of (i) a gene silencing cassette for down-regulating de-etiolated homolog 1, (ii) a gene silencing cassette for down-regulating carotenoid dioxygenase 1B and (iii) a gene silencing cassette for down-regulating cytochrome P450-type monooxygenase.

16. The potato plant of claim 15, wherein the plant's genome comprises all three gene silencing cassettes of (i), (ii) and (iii).

17. The potato plant of claim 16, wherein the potato plant has an increased tuber yield production compared to a wild potato plant of the same variety.

18. The potato plant of claim 17, wherein the plant produces mature tubers having an average size of 26 to 38 mm.

19. A heat-processed product of the potato plant of claim 18, wherein the heat-processed product has superior flavor, texture and appearance compared to a heat-processed product of a wild potato plant of the same variety.

20. The heat-processed product of claim 19, wherein the heat-processed product is a French fry or a roasted potato containing up to 30% of the oil content of a French fry or roasted potato of a wild potato plant of the same variety.

21. A vector comprising (A) an expression cassette for expressing a phytoetene synthase gene; and (B) at least one gene silencing expression cassette selected from the group consisting of (i) a gene silencing cassette for down-regulating de-etiolated homolog 1, (ii) a gene silencing cassette for down-regulating carotenoid dioxygenase 1B and (iii) a gene silencing cassette for down-regulating cytochrome P450-type monooxygenase.

22. The vector of claim 21, wherein the vector comprises all three gene silencing cassettes of (i), (ii) and (iii).


SEQUENCE LISTING (62 sequences)

SEQ ID NO: 1 - DNA, 1093 bp, Solanum tuberosum
caaatatacatatgtatattcactagcattattgggaagacatggggaatgcgaacgccagagaagatggcgccgccgtcgacggcgacggcgacggtgaggtatcgggaagaagatctaatgttgaatctggcatagtagaagatcaccacgctctgacctcgcgagtgccttcggctgacttgatggtcaattctcctccgcagagtccacatcgttcagcttcacctctcttgttcggacctcaggtgccagtagttcctttacaaggaggtgatggcaatcctgttagcaatcaaatgtggggcaatgaatgtgaggatgcctctgatcattcactagagggtggcatacctactttgataacatggagttatggtggtaataatgttgctatccaaggatcttgggacaactggacatcaaggaaaattctccaaagatcaggcaaggactataccgttctcttggtccttccatcgggtatatatcattacaaattcattgtggatggagaagttagatatattccagaacttccatgtgtagcagatgagacaggcgtcgtctttaatcttcttgatgttaatgacaatgttccagagaacctcgaaagtgttgcagagtttgaggccccaccatcacctgattctagctatgcgcaagctttgctggtagatgaggatttcgcaaaggagccagtggcagttccaccccaacttcatctaactgttcttggttctgaaaactcagaagaagcaccttcttctccaaaaccccagcacgtagtacttaatcacctcttcatagagaaaggatgggcttctcaatcggtcgttgctcttggtttaacacatagattccagtccaaatatgtcaccgttgtcctctacaagccactgaagaggtgaccaccccgtgttcacaatcaaatctttccagagctttctcagagtggttgctttaagatttccactgtttatagacctgctcaacgttgaaacttcctttaacctctcttctctgttgtacaatagcttcaagttttatcagcaatgcatcttctcctttcttcaaaaaaaaaaaaaaaaaa

SEQ ID NO: 2 - DNA, 361 bp, Solanum tuberosum
atacctactttgataacatggagttatggtggtaatgacgttgctatccaaggatcttgggacaactggacatcaaggaaaattttccaaagatcaggcaaggactatactgttctcttggtccttccatcgggtatatatcattacaaattcattgtggatggagaagttagatatattccagaacttgcatgtgtagcagatgagacaggcgttgtctttaatctccttgatgttaatgacaatgttccggagaacctcgaaagtgttgcagagtttgaggccccaccatcacctgactctagctatgcgcaagctttgatgggagatgaggatttcgcaaaggagccagtggcagttc

SEQ ID NO: 3 - DNA, 549 bp, Solanum tuberosum
tggcttgtagaggacaacggtgacatatttggactggaatctatgcgttaaaccaagagcaacgacggattgagaagcccatcctttctctataaagaggtgattaagtactacgtgctggggttttgaagaagaaggtgcttcttctgagttttcagaaccaagaacagttagatgaagttgggatggaactgccactggctcctttgcgaaatcctcatctcccatcaaagcttgcgcatagctagagtcaggtgatggtggggcctcaaactctgcaacactttcgaggttctccggaacattgtcattaacatcaaggagattaaagacaacgcctgtctcatctgctacacatgcaagttctggaatatatctaacttctccatccacaatgaatttgtaatgatatatacccgatggaaggaccaagagaacagtatagtccttgcctgatctttggaaaattttccttgatgtccagttgtcccaagatccttggatagcaacgtcattaccaccataactccatgttatcaaagtaggtat

SEQ ID NO: 4 - DNA, 1467 bp, Solanum tuberosum
cgcagtgtgccagggctgtcggcagatggacataaatggcacaccgctcggctcgtggaaagagtatggtcagtttcattgataagtatttactcgtattcggtgtttacatcaagttaatatgttcaaacacatgtgatatcatacatccattagttaagtataaatgccaactttttacttgaatcgccgaataaatttacttacgtccaatatttagttttgtgtgtcaaacatatcatgcactatttgattaagaataaataaacgatgtgtaatttgaaaaccaattagaaaagaagtatgacgggattgatgttctgtgaaatcactggtaaattggacggacgatgaaatttgatcgtccatttaagcatagcaacatgggtctttagtcatcatcattatgttataattattttcttgaaacttgatacaccaactttcattgggaaagtgacagcatagtataaactataatatcaattctggcaatttcgaattattccaaatctcttttgtcatttcatttcctcccctatgtctgcaagtaccaattatttaagtacaaaaaatcttgattaaacaatttattttctcactaataatcacatttaatcatcaacggttcatacacgtctgtcactctttttttattctctcaagcgcatgtgatcataccaattatttaaatacaaaaaatcttgattaaacaattcagtttctcactaataatcacatttaatcatcaacggttcatacacatccgtcactctttttttattctctcaagcgcatgtgatcataccaattatttaaatacaaaaaatcttgattaaacaattcattttctcactaataatcacatttaatcatcaacggtttatacacgtccgccactctttttttattctctcaagcgtatgtgatcatatctaactctcgtgcaaacaagtgaaatgacgttcactaataaataatcttttgaatactttgttcagtttaatttatttaatttgataagaatttttttattattgaatttttattgttttaaattaaaaataagttaaatatatcaaaatatcttttaattttatttttgaaaaataacgtagttcaaacaaattaaaattgagtaactgtttttcgaaaaataatgattctaatagtatattctttttcatcattagatattttttttaagctaagtacaaaagtcatatttcaatccccaaaatagcctcaatcacaagaaatgcttaaatccccaaaataccctcaatcacaagacgtgtgtaccaatcatacctatggtcctctcgtaaattccgacaaaatcaggtctataaagttacccttgatatcagtattataaaactaaaaatctcagctgtaattcaagtgcaatcacactctaccacacactctctagtagagagatcagttgataacaagcttgttaac

SEQ ID NO: 5 - DNA, 359 bp, Solanum tuberosum
ttgattttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaatag

SEQ ID NO: 6 - DNA, 1048 bp, Solanum tuberosum
catatgtatattcactagcattattgggaagacatggggaatgcgaacgccagagaagacggcgccgccggcgacggcgacggtgaggtgtcgggaagaagatctaatgttgaatctggcatagtagaagatcaccacgctctgacctcgcgagtgccttcggctgacttgatggtcaattctcctccgcagagtccacatcgttcagcttcacctctcttgttcggacctcaggtgccagtagttcctttacaaggaggtgatggcaatcctgttagcaatcaaatgtggggcaatgaatgtgaggatgcctctgatcattcactagagggtggcatacctactttgataacatggagttatggtggtaataatgttgctatccaaggatcttgggacaactggacatcaaggaaaattctccaaagatcaggcaaggactataccgttctcttggtccttccatcgggtatatatcattacaaattcattgtggatggagaagttagatatattccagaacttccatgtgtagcagatgagacaggcgtcgtctttaatcttcttgatgttaatgacaatgttccagagaacctcgaaagtgttgcagagtttgaggccccaccatcacctgattctagctatgcgcaagctttgctggtagatgaggatttcgcaaaggagccagtggcagttccaccccaacttcatctaactgttcttggttctgaaaactcagaagaagcaccttcttctccaaaaccccagcacgtagtacttaatcacctcttcatagagaaaggatgggcttctcaatcggtcgttgctcttggtttaacacatagattccagtccaaatatgtcaccgttgtcctctacaagccactgaagaggtgaccaccccgtgttcacaatcaaatctttccagagctttctcagagtggttgctttaagatttccactgtttatagacctgctcaacgttgaaacttcctttaacctctcttctctgttgtacaatagcttcaagttttatcagcaatgcatc

SEQ ID NO: 7 - DNA, 2812 bp, Solanum tuberosum
gcattcttcccacttccagtctttcccatttcatcactatctataatctacaaaaaaaagaaaaatcttcgattctggactgttttttaccagatattcccttgctaattgagtttattttccatggaaggtggtgcaaaatcgaatgatgtatcagcagcacctattgctcaaccactttctgaagaccctactgacattgcatctaatatcaagtatcatgctcaatatactcctcatttttctcctttcaagtttgagccactacaagcatactatgctgctactgctgacagtgttcgtgatcgcttgatcaaacaatggaatgacacctatcttcattatgacaaagttaatccaaagcaaacatactacttatcaatggagtatctccaggggcgagctttgacaaatgcagttggaaacttagacatccacaatgcatatgctgatgctttaaacaaactgggtcagcagcttgaggaggtcgttgagcaggaaaaagatgcagcattaggaaatggtggtttaggaaggctcgcttcatgctttcttgattccatggccacattgaaccttccagcatggggttatggcttgaggtacagatatggactttttaagcagcttatcacaaaggctgggcaagaagaagttcctgaagattggttggagaaatttagtccctgggaaattgtaaggcatgatgttgtctttcctatcaggttttttggtcatgttgaagtcctcccttctggctcgcgaaaatgggttggtggagaggtcctacaggctcttgcatatgatgtgccaattccaggatacagaactaaaaacactaatagtcttcgtctctgggaagccaaagcaagctctgaggatttcaacttgtttctgtttaatgatggacagtatgatgctgctgcacagcttcattctagggctcagcagatttgtgctgttctctaccctggggatgctacagagaatggaaaactcttacggctaaagcaacaattttttctgtgcagtgcatcgcttcaggatattattgccagattcaaagagagagaagatggaaagggttctcaccagtggtctgaattccccaagaaggttgcgatacaactaaatgacacacatccaactcttacgattccagagctgatgcggttgctaatggatgatgaaggacttgggtgggatgaatcttggaatatcactactaggacaattgcctatacgaatcatacagtcctacctgaagcactggaaaaatggtcacaggcagtcatgtggaagctccttcctagacatatggaaatcattgaagaaattgacaaacggtttgttgctacaataatgtcagaaagacctgatcttgagaataagatgcctagcatgcgcattttggatcacaacgccacaaaacctgttgtgcatatggctaacttgtgtgttgtctcttcacatacggtaaatggtgttgcccagctgcatagtgacatcctgaaggctgagttatttgctgattatgtctctgtatggcccaccaagttccagaataagaccaatggtataactcctcgtaggtggatccgattttgtagtcctgagctgagtcatataattaccaagtggttaaaaacagatcaatgggtgacgaacctcgaactgcttgctaatcttcgggagtttgctgataattcggagctccatgctgaatgggaatcagccaagatggccaacaagcagcgtttggcacagtatatactgcatgtgacaggtgtgagcatcgatccaaattccctttttgacatacaagtcaaacgtatccatgaatacaaaaggcagcttctaaatattctgggcgtcatctatagatacaagaagcttaagggaatgagccctgaagaaaggaaaaatacaactcctcgcacagtcatgattggaggaaaagcatttgcaacatacacaaatgcaaaacgaattgtcaagctcgtgactgatgttggcgacgttgtcaatagtgaccctgacgtcaatgactatttgaaggtggtttttgttcccaactacaatgtatctgtggcagagatgcttattccgggaagtgagctatcacaacacatcagtactgcaggcatggaagcaagtggaacaagcaacatgaaatttgcccttaatggatgccttatcattgggacactagatggggccaatgtggaaattagggaggaaattggagaagataacttctttctttttggtgcaacagctgatgaagttcctcaactgcgcaaagatcgagagaatggactgttcaaacctgatcctcggtttgaagaggcaaaacaatttattaggtctggagcatttgggacgtatgattataatcccctccttgaatcactggaagggaactcgggatatggtcgtggagactattttcttgttggtcatgattttccgagctacatggatgctcaggcaagggttgatgaagcttacaaggacaggaaaagatggataaagatgtctatactgagcactagtgggagtggcaaatttagtagtgaccgtacaatttctcaatatgcaaaagagatctggaacattgccgagtgtcgcgtgccttgagcacacttctgaacctggtatctaataaggatctaatgttcattgtttactagcatatgaataatgtaagttcaagcacaacatgctttcttatttcctactgctctcaagaagcagttatttgttgaatatctatattcttctagtaccttcagttactcatttgcttctg

SEQ ID NO: 8 - DNA, 499 bp, Solanum tuberosum
tggagaggtcctacaggctcttgcatatgatgtgccaattccaggatacagaactaaaaacactaatagtcttcgtctctgggaagccaaagcaagctctgaggatttcaacttgtttctgtttaatgatggacagtatgatgctgctgcacagcttcattctagggctcagcagatttgtgctgttctctaccctggggatgctacagagaatggaaaactcttacggctaaagcaacaattttttctgtgcagtgcatcgcttcaggatattattgccagattcaaagagagagaagatggaaagggttctcaccagtggtctgaattccccaagaaggttgcgatacaactaaatgacacacatccaactcttacgattccagagctgatgcggttgctaatggatgatgaaggacttgggtgggatgaatcttggaatatcactactaggacaattgcctatacgaatcatacagtcctacctgaagcacttgaa

SEQ ID NO: 9 - DNA, 568 bp, Solanum tuberosum
gttagaaatcttctctatttttggtttttgtctgtttagattctcgaattagctaatcaggtgctgttatagcccttaattttgagttttttttcggttgtcttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttctccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaatttgagtttttttcgttgttctgattgttgtttttatgaatttgcag

SEQ ID NO: 10 - DNA, 174 bp, Solanum tuberosum
gagcacacttctgaacctggtatctaataaggatctaatgttcattgtttactagcatatgaataatgtaagttcaagcacaacatgctttcttatttcctactgctctcaagaagcagttatttgttgaatatctatattcttctagtaccttcagttactcatttgcttctg

SEQ ID NO: 11 - DNA, 686 bp, Solanum tuberosum
gaaccatgcatctcaatcttaatactaaaaaatgcaacaaaattctagtggagggaccagtaccagtacattagatattatcttttattactataataatattttaattaacacgagacataggaatgtcaagtggtagcggtaggagggagttggttcagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagaccacacatcac

SEQ ID NO: 12 - DNA, 237 bp, Solanum tuberosum
gtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtag

SEQ ID NO: 13 - DNA, 2728 bp, Solanum tuberosum
atgcagagataaaaatatagatcagtctgacaagaaggcaacttctcaaagcttagagagctaccacccgaagatagacagttagttacatgtgtgtatgtgtactgttatagataaaaggagaaatccgaagaagaaagaattttttttgcagatatgtactatcaaggaacctcggataatactaatatacaagctgatcatcaacaacatcataatcatgggaatagtaataataataatattcagacactttatttgatgaaccctaacaattatatgcaaggctacactacttctgacacacagcagcagcagcagttacttttcctgaattcttcaccagcagcaagcaacgcgctttgccatgcgaatatacaacacgcgccgctgcaacagcagcactttgtcggtgtgcctcttccggcagtaagtttgcacgatcagatcaatcatcatggacttttacagcgcatgtggaacaaccaagatcaatctcagcaggtgatagtaccatcgtcgacgggggtttctgccacgtcatgtggcgggatcaccacggacttggcgtctcaattggcgtttcagaggccgattccgacaccacaacaccgacagcagcaacaacagcaaggcggtctatctctaagcctttctcctcagctacaacagcaaattagtttcaataacaatatttcatcctcatcaccaaggacaaataatgttactattaggggaacattagatggaagttctagcaacatggttttaggctctaagtatctgaaagctgcacaagagcttcttgatgaagttgttaatattgttggaaaaagcatcaaaggagatgatcaaaagaaggataattcaatgaataaagaatcaatgcctttggctagtgatgtcaacactaatagttctggtggtggtgaaagtagcagcaggcagaaaaatgaagttgctgtcgagcttacaactgctcaaagacaagaacttcaaatgaaaaaagccaagcttcttgccatgcttgaagaggtggagcaaaggtacagacagtaccatcaccaaatgcaaataattgtattatcatttgagcaagtagcaggaattggatcagccaaatcatacactcaattagctttgcatgcaatttcgaagcaattcagatgcctaaaggatgcaattgctgagcaagtaaaggcgacgagcaagagtttaggtgaagaggaaggcttgggagggaaaatcgaaggctcaagactcaaatttgtggaccatcatctaaggcaacaacgcgcgctgcaacagataggaatgatgcaaccaaatgcttggagaccccaaagaggtttacctgaaagagctatctccgtccttcgtgcttggcttttcgagcattttcttcatccttacccaaaggattcagacaaaatcatgcttgctaagcaaacggggctaacaaggagccaggtgtctaactggttcataaatgctcgagttcgattatggaagccaatggtagaagaaatgtacttggaagaagtgaagaatcaagaacaaaacagtactaatacttcaggagataacaaaaacaaagagaccaatataagcgctccaaatgaagagaaacatccaattattactagcagcttattacaagatggtattactactactcaagcagaaatttctacctcaactatttcaacttcccctactgcaggtgcttcacttcatcatgctcacaatttctccttccttggttcattcaacatggataatactactactactgttgatcatattgaaaacaacgcgaaaaagcaaaggaatgacatgcacaagttttctccaagtagtattctttcatctgttgacatggaagccaaagctagagaatcatcaaataaagggtttactaatcctttaatggcagcatacgcgatgggagattttggaaggtttgatcctcatgatcaacaaatgaccgcgaattttcatggaaataatggtgtctctcttactttaggacttcctccttctgaaaacctagccatgccagtgagccaacaaaattacctttctaatgacttgggaagtaggtctgaaatggggagtcattacaatagaatgggatatgaaaacattgattttcagagtgggaataagcgatttccgactcaactattaccagattttgttacaggtaatctaggaacatgaataccagaaagtctcgtattgatagctgaaaagataaaaggaagttagggatactcttatattgtgtgaggccttctggcccaagtcggaggacccaatttgatacaacctatcataggagaaaagaagtggagactaaattaaagtaacaaaattttaaagcacactttctagtatatatacttcttttttttatagtatagaaaagaagagattttgtgctttagtgtatagatagagtctacttagtataggttatacttctagttccttgagaagattgatacaactagtagtattttttttcttttgggttggcttggagtactattttaagttattggaaactagctatagtaaatgttgtaaagttgtgatattgttcctctcaatttgcatataatttgaaatattttgtacctactagctagtctctaaattatgtttccattgcttgtaattgcaattttatttgaattttgtgctatcattattagattagcaa

SEQ ID NO: 14 - DNA, 1512 bp, Solanum tuberosum
gcaataatggctattgattgtatgatcacaaatgcaaaatctccaatgatagatgaaacaaaacaatttatttttgatgcatcgcatatgaaacgtgagtctaatattcccacacaattcatatggcccgaccacgagaagccttgtgccgtagtacaagaacttcacgtaccccttattgacttaaggggttttctttctggtgactctgacgcggcccaacaagcatctaagcttgttggagaagcgtgtcggagtcatggcttttttcttgtggtgaaccatggagttgatgctaatcttatttctaatgctcatcgttatatggatacgttctttgacttgccgcttttggaaaagcaaaaggctcagaggaaaattggtgagcattgtggttatgctagtagctttactggaaggttttcgtcaaagctaccttggaaagagacactctcttttcgttactctgctgaagaagaatcatctcacatagttgaagatattttcaaaggacattgggtgaaaattttaaccatcttggggaatgtttatcaagaatattgcaattccaagaacacactttctcttgggatcatggaactcttagggatgagcctaggcgtagagaagagccacttcaaagaatttttcgaagaaaatgattcgataatgagactcaactactatccaccatgtcagaaaccggagctcaccttaggaacggggcctcattgtgacccaacatcattaacaattctccatcaagattgtgttggcggacttcaagtttttgtggacgatgaatggcgttccatcagtccaaatttcaatgcatttgtggttaacataggcgacacatttatggcgctatcaaatggaagatacaaaagttgcctgcacagagcagtagtaaacaacaagactcctaggaaatcacttgctttctttctttgtccaaacaaagataaggtggtgagcccaccaaatgaattggtggactccaacaaccctcgaatatatcctgatttcacatggcctactctccttgaatttactcaaaaacattatagagctgatatgaacactcttcaaacattctcaaattgggttcatgatcaacacaatactaaaacacaagtttaagagctacaagattgaagaccttgaattttatttatatatgtacgtgtacacgttctcttagcttctacgaagaatatgacaatgtatggaaattagggggaagaggcataaaggagatgcttctttcgaaaatctcacaagaagaatgaaaatttggaaagaaagaaaaagaggagacatgtccttcttcttcttcttcttttttcccatgaaaagttgagttaaaaaaaaagtaaatatagaggagatagggaaggacaaggaaaggaataaaagtgttgtataacttagttttgtttatttaacttattgaaaaaaatgtagagaagattatgaatagctagctttactggtatccaaaaaaaaaaa

SEQ ID NO: 15 - DNA, 423 bp, Solanum tuberosum
caataatggctattgattgtatgatcacaaatgcaaaatctccaatgatagatgaaaaaaaacaatttatttttgatgcatcgcatatgaaacgtgagtctaatattcccacacaattcatatggcccgaccacgagaagccttgtgccgtagtacaagaacttcacgtaccccttattgacttaagggattttctttctggtgactccgacgcggcccaacaagcatctaagcttgttggagaagcgtgtcggagtcatggcttttttcttgtggtgaaccatggagttgatgctaatcttatttctaatgctcatcgttatatggatacgttctttgacttgccgcttttggaaaagcaaaaggctcagaggaaaattggcgagcattgtggttatgctagtagctttactggaagaatca

SEQ ID NO: 16 - DNA, 405 bp, Solanum tuberosum
agctcaccttaggaacggggcctcattgtgacccaacatcattaacaattctccatcaagattgtgttggcggacttcaagtttttgtggacgatgaatggcgttccatcagtccaaatttcaatgcatttgtggttaacataggcgacacatttatggcgctatcaaatggaagatacaaaagttgcctgcacagagcagtagtaaacaacaagactcctagaaaatcacttgctttctttctttgtccaaacaaagataaggtggtgagcccaccaaatgaattggtggactccaacaaccctcgaatatatcctgatttcacatggcctactctccttgaatttactcaaaaacattatagagctgatatgaacactcttcaaacattctcaaattgggtta

SEQ ID NO: 17 - DNA, 336 bp, Solanum tuberosum
gaagaccttgaaattttatttatatatgtacgtgtacacgttctcttagcttctacgaagaatatgacaatgtatggaaattagggggaagaggcataaaggagatgtttctttcgaaaatttcacaagaagaatgaaaatttggaaagaaagaaaaagaggagacatgttcttcttctttttttccatgaaaagttgagttaaaaaaaaagtaaatatagaagagatagggaaggacaaggaaaggaataaaagtgttgtataacttagttttgtttatttaacttattgaaaaaaatgtagagaagattatgaatagctagctttactggtatc

SEQ ID NO: 18 - DNA, 1307 bp, Solanum tuberosum
ttttctctaagaagaaaattcatgacaatggccattgattgtatggtcatgcctatgatacaaacatcgtccgatgagaaaaacccactcattttcgattcgtccgtgcttaaacatgagtccaacattcctagacaatttatatggccggaccacgagaagcctagtggtggtgttcctgaacttgatgttccacttatcgatttaggggcgttcctttctggtgatccaattgcagctaagcgagaatctaggctcgttgatgaagcatgtaagaaccacggtttctttcttgtgggaaatcatggtgttgacactaatcttatctcacttgcacatcgttatatgaacatgttctttgagttaccactttccaataagcaaatgattcaaaggaagagaggagatcattgtggttatgctagtagctttactgaaagattttcgtctaagttaccttggaaagaaacactttctttttcatattctgccctacaaggctcgtcccacatggttgatcaatattttctaaagacaatgggtgaagactttagccatatcggaaagttttaccaagaatactgcaatgcgatgagcactctctcttctgggatcatggaactcttgggtgaaagcctgggcgtgagcaagaaccatttcaaacaattctttgaagaaaatgaatcgattatgagactcaactattacccaacatgtcaaaaaccagatcttgcgttgggaactggaccacattgtgacccaacatcattaaccatcctccatcaagacagcgtctcagggcttcaagttttcatggacaatcagtggcgctccatcagtccaaatcttagtgcttttgtcgttaacataggcgacacatttatggcgctttcaaatggaaggtataaaagttgtttgcacagagctgtagtaaacaacaagacacctagaaaatctcttgctttttttctttgtcctaagaaggataaggtggtgaggccaccagcagaattggtggactccaataatcctagaatatatccagatttcacatggccaactctccttgagtttacacaaaagcattatagagctgataccaacactctacaattcttctccaattggcttcaacagagaactacagaggtttaaggcatactcaataagtttctagtaggatttctgatatccaatgtaaatcaagaccttagtctttttgtatatatatatatatatatatatatatatatatatatatatattccatgttatttaaattgtttgagttacgaaaaaaaaaaaaa

SEQ ID NO: 19 - DNA, 110 bp, Solanum tuberosum
ggcatactcaataagtttctagtaggatttctgatatccaatgtaaatcaagaccttagtctttttgtatatatatatattccatgttatttaaattgtttgagttacgc

SEQ ID NO: 20 - DNA, 1148 bp, Solanum tuberosum
ccaaagcacatacttatcgatttaaatttcatcgaagagattaatatcgaataatcatatacatactttaaatacataacaaattttaaatacatatatctggtatataattaattttttaaagtcatgaagtatgtatcaaatacacatatggaaaaaattaactattcataatttaaaaaatagaaaagatacatctagtgaaattaggtgcatgtatcaaatacattaggaaaagggcatatatcttgatctagataattaacgattttgatttatgtataatttccaaatgaaggtttatatctacttcagaaataacaatatacttttatcagaacattcaacaaagtaacaaccaactagagtgaaaaatacacattgttctctaaacatacaaaattgagaaaagaatctcaaaatttagagaaacaaatctgaatttctagaagaaaaaaataattatgcactttgctattgctcgaaaaataaatgaaagaaattagacttttttaaaagatgttagactagatatactcaaaagctatcaaaggagtaatattcttcttacattaagtattttagttacagtcctgtaattaaagacacattttagattgtatctaaacttaaatgtatctagaatacatatatttgaatgcatcatatacatgtatccgacacaccaattctcataaaaagcgtaatatcctaaactaatttatccttcaagtcaacttaagcccaatatacattttcatctctaaaggcccaagtggcacaaaatgtcaggcccaattacgaagaaaagggcttgtaaaaccctaataaagtggcactggcagagcttacactctcattccatcaacaaagaaaccctaaaagccgcagcgccactgatttctctcctccaggcgaagatgcagatcttcgtgaagaccctaacggggaagacgatcaccctagaggttgagtcttccgacaccatcgacaatgtcaaagccaagatccaggacaaggaagggattcccccagaccagcagcgtttgattttcgccggaaagcagcttgaggatggtcgtactcttgccgactacaacatccagaaggagtcaactctccatctcgtgctccgtctccgtggtggt

SEQ ID NO: 21 - DNA, 1125 bp, Solanum tuberosum
ttttaaattcactttatatccaagacaatttcagcttaaaaagttttattaatatttacattagttttgttgatgaggatgacaagattttggtcatcaattacatatacccaaattgaatagtaagcaacttaatgtttttcataatgataatgacagacacaaaaaaaacccatttattattcacattgattgatttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgacttttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcatttttttcatcttctatctgatttctattataatttctctcaattgccttcaaatttctctttaag

SEQ ID NO: 22 - DNA, 1233 bp, Zea mays
atggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaaatgagctctcgcaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctag

SEQ ID NO: 23 - DNA, 1650 bp, Artificial Sequence
  Description of Artificial Sequence Synthetic polynucleotide
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgaaaaaaaccgttgtgattggcgcaggctttggtggcctggcgctggcgattcgcctgcaggcggcagggatcccaaccgtactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatga

SEQ ID NO: 24 - DNA, 1497 bp, Solanum lycopersicum
atggaagctcttctcaagccttttccatctcttttactttcctctcctacaccctataggtctattgtccaacaaaatccttcttttctaagtcccaccaccaaaaaaaaatcaagaaaatgtcttcttagaaacaaaagtagtaaacttttttgtagctttcttgatttagcacccacatcaaagccagagtctttagatgttaacatctcatgggttgatcctaattcgaatcgggctcaattcgacgtgatcattatcggagctggccctgctgggctcaggctagctgaacaagtttctaaatatggtattaaggtatgttgtgttgacccttcaccactctccatgtggccaaataattatggtgtttgggttgatgagtttgagaatttaggactggaagattgtttagatcataaatggcctatgacctgtgtgcatataaatgataacaaaactaagtatttgggaagaccatatggtagagttagtagaaagaagctgaagttgaaattgttgaatagttgtgttgagaacagagtgaagttttataaagctaaggtttggaaagtggaacatgaagaatttgagtcttcaattgtttgtgatgatggtaagaagataagaggtagtttggttgtggatgcaagtggttttgctagtgattttatagagtatgacaggccaagaaaccatggttatcaaattgctcatggggttttagtagaagttgataatcatccatttgatttggataaaatggtgcttatggattggagggattctcatttgggtaatgagccatatttaagggtgaataatgctaaagaaccaacattcttgtatgcaatgccatttgatagagatttggttttcttggaagagacttctttggtgagtcgtcctgttttatcgtatatggaagtaaaaagaaggatggtggcaagattaaggcatttggggatcaaagtgaaaagtgttattgaggaagagaaatgtgtgatccctatgggaggaccacttccgcggattcctcaaaatgttatggctattggtgggaattcagggatagttcatccatcaacagggtacatggtggctaggagcatggctttagcaccagtactagctgaagccatcgtcgaggggcttggctcaacaagaatgataagagggtctcaactttaccatagagtttggaatggtttgtggcctttggatagaagatgtgttagagaatgttattcatttgggatggagacattgttgaagcttgatttgaaagggactaggagattgtttgacgctttctttgatcttgatcctaaatactggcaagggttcctttcttcaagattgtctgtcaaagaacttggtttactcagcttgtgtcttttcggacatggctcaaacatgactaggttggatattgttacaaaatgtcctcttcctttggttagactgattggcaatctagcaatagagagcctttga

SEQ ID NO: 25 - DNA, 1891 bp, Solanum tuberosum
cgccacattgtcaactgcattcttcattatctccatttttctctttcatggcttcttctcttcctctttttcaatttccagcacagcattactctaaatctagactcactttctcacctaaattcaatggtagtgcatcaaattttacaattaggtgttcttcaaatggaaaacagcctgagtcggtagatgaaggagtgaaaaaggtggaaaagcttttagaagagaaaaggcgagctgaattatctgctcgtattgcttcaggcgagtttactgttgaacaatctggattctcgtcattgctcaaaaatggtttgtctaaattgggtgtaccaaaggagtttcttgagttcttctctcgacggacgggcgattatcctcgcattccagaggcaaaaggatccatcagtgctattcgggatgagccattcttcatgccgctttatgagctttaccttacttatggcggaattttccggttgatttttggtcccaagtctttcttaatagtttctgatccatcaatagccaaacacatactgaaggataattctaaggcttattctaagggtatcctagctgaaatattggactttgtgatggggaagggacttatacctgcagatggagaaatttggcgcgtcaggcggcgtgccattgtaccagcattgcatcaaaagtacgtagcagctatgattggcttatttggaaaagcaaccgacaggttgtgcaaaaagcttgatgttgctgcaactgatggagaagatgtagagatggaatcactattctcccgtctaacattagacatcattggcaaagctgtatttaattatgattttgactcgttaactgtagacactggtatcgtggaggctgtatatacagtgcttagagaagcagaagatcgcagtattgcaccaattccagtttgggatttgcctatctggaaagatatctctccaaagcttaaaaaggtcaatgcagctctcaagttgattaatgacacattggataatctgattgctatatgtaagaggatggtagacgaagaagagttgcagtttcacgaggaatacatgaatgaaaaagatcctagcatcctccatttcttgttagcatctggagatgaggtctcaagcaagcaacttcgtgatgacctcatgacaatgcttatagccggacatgaaacatctgcggcagtgctaacatggaccttttatctgttgtccaaggaacctagtgtcatggcgaagcttcaagatgaggtcgattcagttctaggggataggttcccaaccattgaagatctaaagaaactcagatacacaactcgtgtgattaatgagtctttaagactatacccacagccaccagtcttgattcgtcgttctattgaagaggacgtagttggaggttacccaattaaaaggggtgaagacattttcatttctgtttggaacttgcatcgatgccccaatcattgggaagaagctgatagattcaatcctgagaggtggccacttgatggacctaacccaaatgagacgaaccaaaatttcagttaccttcccttcggcggaggaccaagaaaatgtgttggagacatgtttgccacatttgagaatttagtggcagttgcaatgcttgttcgacgatttgattttcaaatggctcttggagctcctcctgttaaaatgacaactggggctaccatccacaccacagaaggattaaaaatgactgtaacacgaagaacaagacctccaatagtgcccaaattggagatggcatcattagaagtagatgtcaactcagtgtcaagcgaaagagccaaagctgaagcttctactgttcgaccataatagaactgaatacaagcaacagcagcatg

SEQ ID NO: 26 - DNA, 294 bp, Solanum tuberosum
gcatcgatgcaagttccaaacagaaatgaaaatgtcttcaccccttttaattgggtaacctccaactacgtcctcttcaatagaacgacgaatcaagactggtggctgtgggtatagtcttaaagactcattaatcacacgagttgtgtatctgagtttctttagatcttcaatggttgggaacctatcccctagaactgaatcgacctcatcttgaagcttcgccatgacactaggttccttggacaacagataaaaggtccatgttagcactgctgcagatgtttcatgtcc

SEQ ID NO: 27 - DNA, 536 bp, Solanum tuberosum
tgcctatctggaaagatatctctccaaagcttaaaaaggtcaatgcagctctcaagttgattaatgacacattggataatctgattgctatatgtaagaggatggtagacgaagaagagttgcagtttcacgaggaatacatgaatgaaaaagatcctagcatcctccatttcttgttagcatctggagatgaggtctcaagcaagcaacttcgtgatgacctcatgacaatgcttatagccggacatgaaacatctgcggcagtgctaacatggaccttttatctgttgtccaaggaacctagtgtcatggcgaagcttcaagatgaggtcgattcagttctaggggataggttcccaaccattgaagatctaaagaaactcagatacacaactcgtgtgattaatgagtctttaagactatacccacagccaccagtcttgattcgtcgttctattgaagaggacgtagttggaggttacccaattaaaaggggtgaagacattttcatttctgtttggaacttgcatcgatgc

SEQ ID NO: 28 - DNA, 2117 bp, Solanum tuberosum
taaaagatttctataacatatgaatgtctgaaattccactttacatatttgcttctccttcaccatcaccatgccattttcggtcaccatttcttccttctctcttctcactaaccccaccacccacccccaccaccggaccaccgttctccgcccaaaaaacccacttcaaaatcgttcacaactcaccattaaatcctccattgacaacaagaaaccaccttcaactaaacccacttcatgggtcagtccagattggctaactaaacttaccaggtcacttactttaggccaaaatgatgattctaacatacccattgcgagtgctgagcttgatgatgtttctgaacttttgggtggtgctctttttcttccattgtttagatggatgaatttgtatggacctatttatcgtcttgctgctgggccgaggaattttgttattgttagtgatcctgctattgctaagcatgttttgaagaattatggaaagtatggtaaaggacttgttgctgaggtttctgagtttttgtttggttctggttttgccattgcagaaggtcctctttggacggcaaggcgaagggcagtggttccatctcttcacaagaagtacttgtcagtaatagttgatcgggtcttttgcagatgtgctgagagaatggtggagaaacttttacctgatgcaatttctggctctgcagtgaatatggaggcaaagttttctcaactaacacttgatgttattggtcttgcactcttcaattacaattttgattcccttactactgacagtccagttattgatgcagtatacactgcattaaaagaagcagaactccgttcaactgatctgttgccatattggcagatcaaagccttatgtaagttcatcccacgacaaataaaggctgaaaatgcagtttcactaatcagacaaacagttgaagagctcattgcaaagtgcagagagattgtagaatctgagggtgagaggattaatgaagatgagtacgtgaatgatagagatccaagcattcttcgatttttgcttgctagccgtgaggaggtttcaagtgtacaacttcgagatgatcttctatcaatgctagttgctgggcatgaaaccacaggttcagttttgacttggacggcatacctgctgagtaagaatccttcctctttggaaaaagcacatgaggaagtagatagagttttgggaggacgctctccaacctatgaagacatgaagaatctcaagttcttaacgcggtgcataactgagtcactcagactctatccgcatccacctgtcctgataagaagagctcaagtagctgatgtcctccccgggaattacaaagtcaatgctggtcaagatataatgatttcggtatataacattcatcattcttcagaggtatgggatagagctgaagaatttgatcctgaaagattcgacttggaaggtcccgtcccaaatgaaacgaatactgactttagattcatcccgtttagtggagggccacgaaaatgcgttggtgatcaatttgccttgttggaagctacaattaccctcgcgatatttgtacagaacttctcgttcgagttgattccagatcaaacaattagcatgactactggagcaaccattcatacgacaaacggtttatacatgaaagtgaagcaaagggaaaaagtatctgttttggctgctgcactgtaaattttgtcacagaagaaagttatcttgattctttgaacattatatacatctttggtagactatgagaatctgttctattttttctctggcattattgccttttttctttctttctatatatattagaacatattgctgagcatctttaaaaaataacctctctatatctacgaggtatagggggtaaggctgcgtacacatcaaacctgcccccaagatcctacatgtgagaatacattgaatatgttgttgtatatcagaatatgcctttaaagttgttggagatgtatctattattttcggtttgaagtttgaactctaagaaaataagactttgtttggtagagtgtataaaaataatgttcaatagaatgtattagttatgcttgca

SEQ ID NO: 29 - DNA, 264 bp, Solanum tuberosum
tgggaggacgctctccgacttatgaagacatgaagaatctcaagttcttaacgcggtgcataactgagtcactcagactctatccgcatccacctgtcctaataagaagagctcaagtagctgatgtcctccccgggaattacaaagtcaatgctggtcaagatataatgatttcggtatataacattcatcattcttcagaggtatgggatagagctgaagaatttgatcctgaaaggttcgacttggaaggtcccgtcccaa

SEQ ID NO: 30 - DNA, 2045 bp, Solanum tuberosum
caattaaaagatgtattcaactgtgttttacacttcagttcatccttccacttcaattttatcaagaaagcagctacctttattgatttccaaggacttccctacagagttatatcattctttaccttgtaggagcttggaaaatgggcatatcaagaaggttaaaggagtaaaagtaaaagccacaatagctgaagctccggttactcctacagagaagagtgactctggggttaacggtgatttgaaggttccacagaagaagttgaaagtacttgttgcgggtggtgggattggagggttagtttttgctttagcagcaaagaaaaaggggtttgatgtgttggtgtttgagagggatttaagtgctatcagaggagagggacaatatagaggtccaattcagatacagagcaatgcattggctgctttggaagcaattgatatggatgttgctgaagacatcatgaatgctggctgcatcactggtcaaaggattaatggcttggttgatggtatttctggcaactggtattgcaagtttgatacgttcactccagcagtggaacgtggacttcccgtgacaagagtcatcagccgcatgactttgcagcagatccttgcacgtgctgttggggaggatacaattatgaatgaaagtaatgttgtagactttgaggatgatggggagaaggttagtgtggttcttgagaatggacaacgatttacaggtgatcttctggttggtgctgatggcataaggtctaaggtacggactaatctttttggacccagtgaagttacttactctggctacacttgttatactggaattgcagattttgttcctgctgatattgatacagttgggtaccgagtctttttgggccacaaacagtactttgtttcttcagatgtggggggaggcaagatgcagtggtatgcattttacaatgaaccagctggtggtgtggatgctccaaatggtaaaaaggaaagattgcttaaaatatttgggggatggtgtgacaatgtcatagacctattaattgccacagatgaagatgcaattcttcgtcgtgacatctatgatagaccaccaacttttagttggggaagaggtcgtgctacattgcttggggactctgtccatgctatgcagcctaatttgggtcaagggggatgcatggccatagaggatagctatcaactagcactggaacttgacaaagcgtgtagtcgaagtgccgagtcaggaagccctgtggatatcatctcatctttaaggagctatgaaagtgctagaaaacttcgagttggagtcatccatggactggctagaatggctgcaatcatggcatctacttacaaagcttatcttggcgtcggacttagtccactatcatttttgacgcagtatagaataccacatcctggaagagttggtggaagagtatttattgacttgggaatgcctctgatgttaagttgggttctaggaggcaatggggacaagcttgaaggcagaataaaacattgcaggctatctgagaaagcaaatgaccaattgagaaaatggtttgaagatgatgatgcattagagcgtgctactgatgcagagtggttacttttacctgcggggaatggcacttctggtttagaagctattgttttaagcagagatgaggatgtcccttgcactatcgggtctgtctcacatacaaacattcctggaaaatcagtagttttacctttgccacaggtgtctgaaatgcacgcccgaatatcctgcaaagatggagcattttttgtaactgatttacaaagcgaacatggcacctgggttacagataatgaaggcagaagataccggacgtctccaaacttccctacacgttttcatccatcagatgttatcgaatttggttctgataaggcagcatttcgtgtaaaggcaatgaaatttcctccaaaaactactactgaaaggaaggaagagcgtgaagcagtgggggcagcgtaaagcttgacctgatataaacagg

SEQ ID NO: 31 - DNA, 302 bp, Solanum tuberosum
atcagtagttttacctttgccacaggtgtctgaaatgcacgcccgaatatcctgcaaagacggagcattttttgtaactgatttacaaagcgaacatggcacctgggttacagataatgaaggcagaagataccggacgtctccaaacttccctacacgttttcatccatcagatgttatcgaatttggttctgataaggcagcatttcgtgtaaaggcaatgaaatttcctccaaaaactactgaaaggaaggaagagcgtgaagctgtgggggcagcgtaaagcttgacctgatataaac

SEQ ID NO: 32 - DNA, 1113 bp, Solanum tuberosum
atggcaacttcaattttcctctcacaccctttttctcctttattatctaaacaacaccataaaattccaagtcccaaacaaaccatagccatagcatatcactcccccaacaaacctaccaccaagactccatttttaccatttcccacttccttttttccatttccctcgaaccccagaaaggaattttggccaatttcagtgggaagaacacaaacagatgaaaaagatgaaatcttggtggtgggtgaagattctgctgaatttgagttatccaaacaaaagatttcatcttgggtttattttacagggattcttggtgttgtgctttatgttcttaatgttgtttggattgacaattctactggatttggaaaatcattcattgattctgtttctagtatttcagatagccctgagattgtaatgctttcccttaccttgattttcgctatagtccacagtggtcttgctagtcttagagacaaaggtgaggaactcattggagagcgtgcttttcgtgtattgtttgctggggtatctctgccattggcagtcagcacaattgtgtatttcattaaccaccgatacgatggagtgcagttatggcaattaaacagcgttgctggaattcatgaactagtttggatttctaactttatttccttcttcttcctatacccgtcgacattcaatttactagaggtagcggctgttgacaagcccaagatgcatctttgggaaactgggattatgaggattaccaggcatccacagctggttgggcaggttatatggtgcttagctcacacgctgtggattgggaattcagttgcagtggcagcctcagtaggtttgataggacatcatctgtttggtgcctggaatggggaccggaggttagccatacgatatggtgaggcttttgaagtcgtgaagaacagaacgagtatcattccatttgctgctattcttgatggtcgtcaaaagttgcctgaagattattacaaggaatttatcagattgccatacttatcgataacagcattgacattaggtgcttacttcctccaccccattatgcaagctgccagttatcggctacactggtag

SEQ ID NO: 33 - DNA, 157 bp, Solanum tuberosum
ctgattcctccataagaccttgactgatgttacctcattggttctcaacttgcgaacctgacgctccagaatctgaacgggaatctcctcaaaagataagatatccttaataccaatgtctttagtcggtataataagagaaggatcacccatacac

SEQ ID NO: 34 - DNA, 1497 bp, Solanum tuberosum
atggaagctcttctcaagccttttccatctcttttactttcctctcctacaccctacaggtctattgtccaacaaaatccctcttttctaagtcccaccaccaaaaaaaaatcaagaaaatgtcttcttagaaacaaaagtagtaaacttttttgtagctttcttgatttagcacccacatcaaagccagagtctttagatgttaacatctcatgggttgatcctaattcaggtcgggctcaattcgacgtgatcatcatcggagctggccctgctggtctcaggctagctgaacaagtttctaaatatggtattaaggtatgttgtgttgacccttcaccactttccatgtggccaaataattatggtgtttgggttgatgagtttgagaatttaggattggaagattgtttagatcataaatggcctatgacttgtgtgcatataaatgatcataaaactaagtatttgggaagaccatatggtagagtaagtagaaagaagctgaagttgagattattgaatagttgtgttgaaaacagggtgaagttttataaagctaaggtttggaaagtggaacatgaagaatttgagtcttcaattgtttgtgatgatggtaagaagataagaggtagtttggttgtggatgcaagtggttttgctagtgattttatagagtatgacaagccaagaaaccatggttatcaaattgctcatggggttttagtagaagttgataatcatccatttgatttggataaaatggtgcttatggattggagggattctcatttaggtaatgagccatatttaagggtgaataatgctaaagaaccaacattcttgtatgcaatgccatttgataagaatttggttttcttggaggagacttctttggtgagtcgtcctgtgttatcgtatatggaagtaaaaagaaggatggtggcaagattaaggcatttggggatcaaagtgagaagtgttattgaggaagagaaatgtgtgatccctatgggaggaccacttccgcggattcctcaaaatgttatggctattggtgggaattcggggatagttcatccatcgacagggtacatggtggctaggagcatggctttggcaccagtactggctgaagccatcgtcgaggggcttggctcaacaagaatgataagagggtctcaactttaccatagagtttggaatggtttgtggcctttggatagaagatgtgttagagaatgttattcatttgggatggagacattgttgaagcttgatttgaaagggactaggagattgtttgatgctttctttgatcttgatcctaaatactggcaagggttcctttcttcaagattgtctgtcaaagaacttgctttactcagcttgtgtctttttggacatggctcaaatttgactaggttggatattgttacaaaatgtcctgttcctttggttagactgattggcaatctagcaatagagagcctttga

SEQ ID NO: 35 - DNA, 214 bp, Solanum tuberosum
catggggtgccgtatctagtttatataataaatatcgtttctagtctatctcttctgatgctaaataaagtcagtgattattttttaattttttctactaggtaatgtaaaattcttatgttaaccaaataaattgagacaaattaattcagttaaccagagttaagagtaaagtactattgcaagaaaatatcaaaggcaaaagaaaagatca

SEQ ID NO: 36 - DNA, 1148 bp, Solanum tuberosum; identical to SEQ ID NO: 20

SEQ ID NO: 37 - DNA, 418 bp, Solanum tuberosum
cttccagtaaagctactagcataaccacaatgctcgccaattttcctctgagccttttgcttttccaaaagcggcaagtcaaagaacgtatccatataacgatgagcattagaaataagattagcatcaactccatggttcaccacaagaaaaaagccatgactccgacacgcttctccaacaagcttagatgcttgttgggccgcgtcggagtcaccagaaagaaaatcccttaagtcaataaggggtacgtgaagttcttgtactacggcacaaggcttctcgtggtcgggccatatgaattgtgtgggaatattagactcacgtttcatatgcgatgcatcaaaaataaattgttttttttcatctatcattggagattttgcatttgtgatcatacaatcaatagccattattg

SEQ ID NO: 38 - DNA, 157 bp, Solanum tuberosum; identical to SEQ ID NO: 33

SEQ ID NO: 39 - DNA, 418 bp, Solanum tuberosum
caataatggctattgattgtatgatcacaaatgcaaaatctccaatgatagatgaaaaaaaacaatttatttttgatgcatcgcatatgaaacgtgagtctaatattcccacacaattcatatggcccgaccacgagaagccttgtgccgtagtacaagaacttcacgtaccccttattgacttaagggattttctttctggtgactccgacgcggcccaacaagcatctaagcttgttggagaagcgtgtcggagtcatggcttttttcttgtggtgaaccatggagttgatgctaatcttatttctaatgctcatcgttatatggatacgttctttgacttgccgcttttggaaaagcaaaaggctcagaggaaaattggcgagcattgtggttatgctagtagctttactggaag

SEQ ID NO: 40 - DNA, 1125 bp, Solanum tuberosum; identical to SEQ ID NO: 21

SEQ ID NO: 41 - DNA, 214 bp, Solanum tuberosum; identical to SEQ ID NO: 35

SEQ ID NO: 42 - DNA, 2260 bp, Solanum tuberosum
aagtgtctgagacaaccaaaactgaaagtgggaaaccaaactctaagtcaaagactttatatacaaaatggtataaatataattatttaatttactatcgggttatcgattaacccgttaagaaaaaacttcaaaccgttaagaaccgataacccgataacaaaaaaaatctaaatcgttatcaaaaccgctaaactaataacccaatattgataaaccaataactttttttattcgggttatcggtttcagttctgtttggaacaatcctagtgtcctaattattgttttgagaaccaagaaaacaaaaacttacgtcgcaaatatttcagtaaatacttgtatatctcagtgataattgatttccaacatgtataattatcatttacgtaataatagatggtttccgaaacttacgcttcccttttttcttttgcagtcgtatggaataaaagttggatatggaggcattcccgggccttcaggtggaagagacggagctgcttcacaaggagggggttgttgtacttgaaaatgggcatttattgttcgcaaacctatcatgttcctatggttgtttatttgtagtttggtgttcttaatatcgagtgttctttagtttgttccttttaatgaaaggataatatctgtgcaaaaataagtaaattcggtacataaagacatttttttttgcattttctgtttatggagttgtcaaatgtgaatttatttcatagcatgtgagtttcctctcctttttcatgtgcccttgggccttgcatgtttcttgcaccgcagtgtgccagggctgtcggcagatggacataaatggcacaccgctcggctcgtggaaagagtatggtcagtttcattgataagtatttactcgtattcggtgtttacatcaagttaatatgttcaaacacatgtgatatcatacatccattagttaagtataaatgccaactttttacttgaatcgccgaataaatttacttacgtccaatatttagttttgtgtgtcaaacatatcatgcactatttgattaagaataaataaacgatgtgtaatttgaaaaccaattagaaaagaagtatgacgggattgatgttctgtgaaatcactggtaaattggacggacgatgaaatttgatcgtccatttaagcatagcaacatgggtctttagtcatcatcattatgttataattattttcttgaaacttgatacaccaactttcattgggaaagtgacagcatagtataaactataatatcaattctggcaatttcgaattattccaaatctcttttgtcatttcatttcctcccctatgtctgcaagtaccaattatttaagtacaaaaaatcttgattaaacaatttattttctcactaataatcacatttaatcatcaacggttcatacacgtctgtcactctttttttattctctcaagcgcatgtgatcataccaattatttaaatacaaaaaatcttgattaaacaattcagtttctcactaataatcacatttaatcatcaacggttcatacacatccgtcactctttttttattctctcaagcgcatgtgatcataccaattatttaaatacaaaaaatcttgattaaacaattcattttctcactaataatcacatttaatcatcaacggtttatacacgtccgccactctttttttattctctcaagcgtatgtgatcatatctaactctcgtgcaaacaagtgaaatgacgttcactaataaataatcttttgaatactttgttcagtttaatttatttaatttgataagaatttttttattattgaatttttattgttttaaattaaaaataagttaaatatatcaaaatatcttttaattttatttttgaaaaataacgtagttcaaacaaattaaaattgagtaactgtttttcgaaaaataatgattctaatagtatattctttttcatcattagatattttttttaagctaagtacaaaagtcatatttcaatccccaaaatagcctcaatcacaagaaatgcttaaatccccaaaataccctcaatcacaagacgtgtgtaccaatcatacctatggtcctctcgtaaattccgacaaaatcaggtctataaagttacccttgatatcagtattataaaactaaaaatctcagctgtaattcaagtgcaatcacactctaccacacactctctagtagagagatcagttgataacaagcttgttaac

SEQ ID NO: 43 - DNA, 302 bp, Solanum tuberosum
gtttatatcaggtcaagctttacgctgcccccacagcttcacgctcttccttcctttcagtagtttttggaggaaatttcattgcctttacacgaaatgctgccttatcagaaccaaattcgataacatctgatggatgaaaacgtgtagggaagtttggagacgtccggtatcttctgccttcattatctgtaacccaggtgccatgttcgctttgtaaatcagttacaaaaaatgctccgtctttgcaggatattcgggcgtgcatttcagacacctgtggcaaaggtaaaactactgat

SEQ ID NO: 44 - DNA, 264 bp, Solanum tuberosum
ttgggacgggaccttccaagtcgaacctttcaggatcaaattcttcagctctatcccatacctctgaagaatgatgaatgttatataccgaaatcattatatcttgaccagcattgactttgtaattcccggggaggacatcagctacttgagctcttcttattaggacaggtggatgcggatagagtctgagtgactcagttatgcaccgcgttaagaacttgagattcttcatgtcttcataagtcggagagcgtcctccca

SEQ ID NO: 45 - DNA, 294 bp, Solanum tuberosum; identical to SEQ ID NO: 26

SEQ ID NO: 46 - DNA, 535 bp, Solanum tuberosum
tgcctatctggaaagatatctctccaaagcttaaaaaggtcaatgcagctctcaagttgattaatgacacattggataatctgattgctatatgtaagaggatggtagacgaagaagagttgcagtttcacgaggaatacatgaatgaaaaagatcctagcatcctccatttcttgttagcatctggagatgaggtctcaagcaagcaacttcgtgatgacctcatgacaatgcttatagccggacatgaaacatctgcggcagtgctaacatggaccttttatctgttgtccaaggaacctagtgtcatggcgaagcttcaagatgaggtcgattcagttctaggggataggttcccaaccattgaagatctaaagaaactcagatacacaactcgtgtgattaatgagtctttaagactatacccacagccaccagtcttgattcgtcgttctattgaagaggacgtagttggaggttacccaattaaaaggggtgaagacattttcattttgtttggaacttgcatcgatgc

SEQ ID NO: 47 - DNA, 264 bp, Solanum tuberosum; identical to SEQ ID NO: 29

SEQ ID NO: 48 - DNA, 302 bp, Solanum tuberosum; identical to SEQ ID NO: 31

SEQ ID NO: 49 - DNA, 686 bp, Solanum tuberosum; identical to SEQ ID NO: 11

SEQ ID NO: 50 - DNA, 1467 bp, Solanum tuberosum; identical to SEQ ID NO: 4

SEQ ID NO: 51 - DNA, 1497 bp, Solanum tuberosum; identical to SEQ ID NO: 34

SEQ ID NO: 52 - DNA, 359 bp, Solanum tuberosum; identical to SEQ ID NO: 5

SEQ ID NO: 53 - DNA, 476 bp, Solanum tuberosum
caacatccacgccctcgagcaactaaggtgtacaaagaataaagcaaccaatttctctcaatttgttaagaagatgatggcttccttgccttttagttgtcagtctcagagtccttccccatattttgaccaatctctcttcaggtttgatgagaagcttatttcagcgattgaccgacatagacagtctactgaccatccaatcaaattcatttctagaagacaacccaatatcctgaaattcaaaatgaagccaggacccgaagctggcagcacggatgggcgaactaagaagatctgttccttcctcttccacccaatattaccccttgcactttctgttcaacaaaccttgtttctgcaggcatcagttgtaaatatccattttcgtcgataatgtaaaaacttttttatatgttacctatttgtttataaatttcgctaataacctctagattgaaatcaacctagaaatc

SEQ ID NO: 54 - DNA, 312 bp, Solanum tuberosum
cagcgattgaccgacatagacagtctactgaccatccaatcaaattcatttctagaagacaacccaatatcctgaaattcaaaatgaagccaggacccgaagctggcagcacggatgggcgaactaagaagatctgttccttcctcttccacccaatattaccccttgcactttctgttcaacaaaccttgtttctgcaggcatcagttgtaaatatccattttcgtcgataatgtaaaaacttttttatatgttacctatttgtttataaatttcgctaataacctctagattgaaatcaacctagaaatc

SEQ ID NO: 55 - DNA, 1855 bp, Solanum tuberosum
  modified_base (1655)..(1655): a, c, t, g, unknown or other
atgttcaaaagcaacaatgttaccgccaggctttttgagcgccagatttgcacccctgctcctggcaccagcatccatcgtgccagaagattttatgagaacattgtaccaagttataccatatacgatgttgaatgtcccgaccattcatttcgcaagttcacggatgatggtctatatcttgtaagtttcagccgaaatcatcaggatctggttgtttatagaccaacatggttgacattttcctgcaaagaagaagattgtgatactcatgatcttcctttgaaagctagaaagtttgagagcttcttcacgcagttgtacagtgttactcttgcttctagtggggaacttatatgcaaagatttctttctctatatggagaacaaccaatttggactctttgcaacttcaactgcacaaattcatgatgcacctcctactggaggggcaattcagggagtcccttcagttgaaaaaataactttccaccttttgaggttggaggatggagcgatacttgacgagagggtttttcacaatgattatgttaatttggcacatagcattggtgctttcttgtatgatgatttgcttgctatagtgtctcttcgttatcaaagaatacacatccttcagatcagagattctggagatcttgttgatgtacgagcaattggggaattctgccgtgaagatgatgaactttttctcaattccaattcacaggtgcttgtaaatcatgttggaaatggttttcatcatagtctgcctgaatcggaaacttctttcctgagcggtataaagcaacggctgctttcatttatatttcgaggtatatggaatgaagaaactgaccaaaccatgcgagtgcagtgcctgaagaagaagttttacttccactttcaagattacattgacttgattatctggaaggtgcaatttttggaccgacatcacctgttgatcaagtttggcagtgttgatggtggggtatctcgaaatgctgacagccatccttctttttttgctgtttacaatatggagactactgaaattgttgcattttatcagaactcagccgatgagctttatttcttgttcgagctgttcagcgaccattttcatgtttcatccaaaagttcattacatatgaactttatgtcctcacactcaaacaacatccacgccctcgagcaactaaggtgtacaaagaataaagcaaccaatttctctcaatttgttaagaagatgatggcttccttgccttttagttgtcagtctcagagtccttccccatattttgaccaatctctcttcaggtttgatgagaagcttatttcagcgattgaccgacatagacagtctactgaccatccaatcaaattcatttctagaagacaacccaatatcctgaaattcaaaatgaagccaggacccgaagctggcagcacggatgggcgaactaagaagatctgttccttcctcttccacccaatattaccccttgcactttctgttcaacaaaccttgtttctgcaggcatcagttgtaaatatccattttcgtcgataatgtaaaaacttttttatatgttacctatttgtttataaatttcgctaataacctctagattgaaatcaacctagaaatcncaaattgattataagagacctgtagatgctagtctcttcgactcctacattttctttgctncaagcatcaaacaaatgcttgatagagtgccaagagggttagtatggtcataaggatttgttcttctgtaagttgtttgttacagcttctcttcccacttattgatggacattcagatatttatttgtgatgtagaaaa

SEQ ID NO: 56 - DNA, 1857 bp, Solanum tuberosum
ggaaaatggggatgaaagaagaaaatggagtggcgagaattgaaggaggagtagtggtggttgatccaaagccacaaaatggagtcgctgcaaaagcgatagactgggtagaatgggcgattatcaaattgatgaacgattctaccaagccactcccctttcttcaggggaattttgcacctactgatgaaactcctccacttaaacaccttcccgttataggccatcttccggaatgcctgaatggtgagtttgttagggttggtccaaatcctaaatttgctccagttgctggatatcattggtttgatggagatggcatgattcatggcttgcaaattaaggatggaaaagcaacatatgtctcacgttttgtgaggacatcacgtcttaagcaagaagagttctttggaggagctaagtttatgaagattggagatcttaaagggctgttcgggttgttctcagtatatatttacatgctcagggaaaagctgaaagttttggacacttcctatggacatggcacagctaatacagctatgatatatcaccacgggaagcttttggctcttcatgagggtgataaaccatatgtagttaagattctggaggatggagatctgcaaacgcttggcatgctggattacgataaaaggctgcaacatccctttactgctcacccgaaggttgaccctgtaactggggaaatgtttacctttggctactcacagacaccacctttcgctacatatagagtcatatccaaggatggtgtcatgcaagatccagttccaataacgataccagcatctgttatgatgcacgattttgctattactgaaaattatgcaattatgatggatcttccattgtacttcagaccaaaggaaatggtgaaaaagaaacagctggcatacagctttgaccccacaaagaaggctcgttttggagttcttccacgctatgcaaagaacgaatccctaatcaagtggttcgagcttcctaactgcttcatattccacaacgccaatgcttgggaggagggagatgacgtggtcttgatcacttcccgcctgcagaatccagatctagacgcgattaagggaactgaaaaagaagaacagcgtgacggtttcacaaatgagttgtatgagatgaggttcaatatgaagagtggtttagcatcacagaagaaactgtcagaggctgctgttgattttccacggatcaacgagaactacactggaaggaagcaacgctatgtatatggaaccattttaaacaacgttgcccagatcacaggagttgtcaaatttgatttgcatgcggaaccagagactggaaaaacaaagcttgaagtaggtggaaatgttcctggaattttcgaccttggacctggaagatttggctcagaggcaatatttgttccccgtcaacctgggactgaatgtgaagaggatgacggctacttaatattgtttgtacatgatgagaacactggaaagtcatcagtgaatgtaattgatgcgaaaacaatgtcagctgaacctgtggcagtcgttgaattacctaaaagagttccatttggattccacgccttctttgtcacagaggaacaaattcaagagcaagccaaaatgtgagaagagtggatcagaaagtcagtctacgaacaacatgcaactctgttttcaattggcatcatcaatattattctgtgatatatattgtaattatatgtttatagtacctgttatgagcaggtggctactggtgactataacatagtaataaatgtatgtcaagttttctgtgtgtatctgcactcatttcatattatacaaatgattatgg

SEQ ID NO: 57 - DNA, 449 bp, Solanum tuberosum
atggggatgaaagaagaaaatggagtggcgagaattgaaggaggagtagtggtggttgatccaaagccacaaaatggagtcgctgcaaaagcgatagactgggtagaatgggcgattatcaaattgatgaacgattctaccaagccactcccctttcttcaggggaattttgcacctactgatgaaactcctccacttaaacaccttcccgttataggccatcttccggaatgcctgaatggtgagtttgttagggttggtccaaatcctaaatttgctccagttgctggatatcattggtttgatggagatggcatgattcatggcttgcaaattaaggatggaaaagcaacatatgtctcacgttttgtgaggacatcacgtcttaagcaagaagagttctttggaggagctaagtttatgaagattggagatcttaaagggctgtt

SEQ ID NO: 58 - DNA, 284 bp, Solanum tuberosum
aattttgcacctactgatgaaactcctccacttaaacaccttcccgttataggccatcttccggaatgcctgaatggtgagtttgttagggttggtccaaatcctaaatttgctccagttgctggatatcattggtttgatggagatggcatgattcatggcttgcaaattaaggatggaaaagcaacatatgtctcacgttttgtgaggacatcacgtcttaagcaagaagagttctttggaggagctaagtttatgaagattggagatcttaaagggctgtt

SEQ ID NO: 59 - DNA, 549 bp, Solanum tuberosum
atacctactttgataacatggagttatggtggtaataatgttgctatccaaggatcttgggacaactggacatcaaggaaaattctccaaagatcaggcaaggactataccgttctcttggtccttccatcgggtatatatcattacaaattcattgtggatggagaagttagatatattccagaacttccatgtgtagcagatgagacaggcgtcgtctttaatcttcttgatgttaatgacaatgttccagagaacctcgaaagtgttgcagagtttgaggccccaccatcacctgattctagctatgcgcaagctttgctggtagatgaggatttcgcaaaggagccagtggcagttccaccccaacttcatctaactgttcttggttctgaaaactcagaagaagcaccttcttctccaaaaccccagcacgtagtacttaatcacctcttcatagagaaaggatgggcttctcaatcggtcgttgctcttggtttaacacatagattccagtccaaatatgtcaccgttgtcctctacaagcca

SEQ ID NO: 60 - DNA, 699 bp, Solanum tuberosum
acgacctcgtttgttcggaacacaattttttctttttcattttttattctccacagaaacttttctttttcatttgatagtataaaaaattcaaaaaaatatttttgtcgtatttccctcattattaattgttgataataatacttggaggctatcgctatcattgtgctctcaaaccaacgtgggcacacacctaaagaagataatatatgcacaaaaaagagtacattttatacacattcataaatttagttaatctacaccttccattttgtacttatcctttatcaaccattctgatctctccatgtcatcactatatatcctctaaattttccttttatatttttccaatttccatctccatccttttccgctcgccctttaattgagagtctttccataacaacttttctatttctcaatatataagaataagatctgcatatatttcactacatttattgtattatttcatagattaattgagatgctcgtaagctcaccctccaatcgaaagtctttccgaaataacttttttatttctcaacagataagaatgatctgcatatatttcattgcatttgttatattatttcgtagattaatcgaggtgctagtaagcaaaaagtagaaggaaaaagaaagtcaattgagggcattattgtaaataagtccaatagtgtgccttatctttta

SEQ ID NO: 61 - DNA, 157 bp, Solanum tuberosum
gtgtatgggtgatccttctcttattataccgactaaagacattggtattaaggatatcttatcttttgaggagattcccgttcagattctggagcgtcaggttcgcaagttgagaaccaatgaggtaacatcagtcaaggtcttatggaggaatcag

SEQ ID NO: 62 - DNA, 997 bp, Solanum tuberosum
gttggaaaaagcatcaaaggagatgatcaaaagaaggataattcaatgaataaagaatcaatgcctttggctagtgatgtcaacactaatagttctggtggtggtgaaagtagcagcaggcagaaaaatgaagttgctgtcgagcttacaactgctcaaagacaagaacttcaaatgaaaaaagccaagcttcttgccatgcttgaagaggtggagcaaaggtacagacagtaccatcaccaaatgcaaataattgtattatcatttgagcaagtagcaggaattggatcagccaaatcatacactcaattagctttgcatgcaatttcgaagcaattcagatgcctaaaggatgcaattgctgagcaagtaaaggcgacgagcaagagtttaggtgaagaggaaggcttgggagggaaaatcgaaggctcaagactcaaatttgtggaccatcatctaaggcaacaacgcgcgctgcaacagataggaatgatgcaaccaaatgcttggagaccccaaagaggtttacctgaaagagctatctccgtccttcgtgcttggcttttcgagcattttcttcatccttacccaaaggattcagacaaaatcatgcttgctaagcaaacggggctaacaaggagccaggtgtctaactggttcataaatgctcgagttcgattatggaagccaatggtagaagaaatgtacttggaagaagtgaagaatcaagaacaaaacagtactaatacttcaggagataacaaaaacaaagagaccaatataagcgctccaaatgaagagaaacatccaattattactagcagcttattacaagatggtattactactactcaagcagaaatttctacctcaactatttcaacttcccctactgcaggtgcttcacttcatcatgctcacaatttctccttccttggttcattcaacatggataatactactactactgttgatcatattgaaaacaacgcgaaaa